(19) United States
(12) Patent Application Publication
(10) Pub. No.: US 2026/0144999 A1
(43) Pub. Date: May 28, 2026

(54) NEURONAVIGATED TRANSCRANIAL BRAIN ENERGY DELIVERY AND DETECTION SYSTEM AND METHOD

(71) Applicant: Ampa Inc., San Diego, CA (US)

(72) Inventors: Donald A. VAUGHN, San Diego, CA (US); Jonathan A.S. DOWNAR, Carrying Place (CA)

(21) Appl. No.: 19/367,618

(22) Filed: Oct. 23, 2025

Related U.S. Application Data
(63) Continuation of application No. 19/119,714.
Application No. 19/119,714 is a continuation of application No. 17/964,686, filed on Oct. 12, 2022, now Pat. No. 11,730,969.
Application No. 17/964,686 is a continuation of application No. 18/212,024, filed on Jun. 20, 2023.
Application No. 18/212,024 is a continuation of application No. 18/212,055, filed on Jun. 20, 2023.
Application No. 18/212,055 is a continuation of application No. 18/212,081, filed on Jun. 20, 2023.

(51) Int. Cl. A61N 2/00; A61B 5/055; A61B 5/291; A61B 5/369; A61N 1/36; A61N 2/02

(52) U.S. Cl. CPC A61N 2/006; A61B 5/055; A61B 5/291; A61B 5/369; A61N 1/36025; A61N 2/02

(57) ABSTRACT

A neuronavigated transcranial brain stimulation system including i) a treatment device including an energy delivery and/or detection instrument configured to be placed over a target brain region of an individual's head for treatment; ii) a generator for energizing the energy delivery and/or detection instrument; and iii) one or more imaging devices incorporated into the energy delivery and/or detection instrument and configured to permit direct visualization of a position of the energy delivery and/or detection instrument on an individual's head, over the target brain region.

### CROSS REFERENCE TO RELATED APPLICATIONS

This application is a continuation application of U.S. patent application Ser. No. 19/119,714, filed Apr. 9, 2025, which is a 371 US National Phase Application of PCT International Patent Application Serial No. PCT/US 2023/034693, filed Oct. 6, 2023, which claims priority to U.S. patent application Ser. No. 17/964,686, filed Oct. 12, 2022, now U.S. Pat. No. 11,730,969, U.S. patent application Ser. No. 18/212,024, filed Jun. 20, 2023, U.S. patent application Ser. No. 18/212,055, filed Jun. 20, 2023, and U.S. patent application Ser. No. 18/212,081, filed Jun. 20, 2023, the contents of which are incorporated herein by reference.

### BACKGROUND OF THE DISCLOSURE

The present disclosure relates to neuronavigated transcranial brain energy delivery and/or detection systems and methods. The disclosure has particular utility for transcranial magnetic stimulation (TMS), i.e., methods and apparatus for positioning a transcranial magnetic stimulation device properly on the head of patients so as to deliver magnetic stimulation to a specific brain region. The present disclosure also advantageously can be used to provide neuronavigation for devices that stimulate, radiate, or sense energy emissions from the brain, such as focused ultrasound (FUS), transcranial electrical stimulation (TES), electroencephalography (EEG), magnetoencephalography (MEG) near infrared spectroscopy (NIRS) and gamma knife radiation, and the like each of which techniques has an effective field of stimulation, radiation, or sensation which is relatively small. Thus, each require systems, i.e., apparatus and processes to assist in precise neuronavigation.

Neuronavigated transcranial systems and methods for applying stimulation to target brain regions of a patient for disorders such as depression, Obsessive Compulsive Disorder (OCD), depression with comorbid anxiety, and nicotine addiction, and as well as Major Depression Disorder (MDD), bipolar disorder, Post-Traumatic Stress Disorder (PTSD), eating disorders, personality disorders, alcohol, drug and other substance use disorders, gambling, smoking cessation, as well as neurological illnesses including Alzheimer's Disease, mild cognitive impairment and other dementias, migraine headaches, movement disorders such as Parkinson's Disease, Asperger's Syndrome, Multiple Sclerosis (MS), ALS, Tourette's Syndrome, blepharospasm, stroke, autism, tinnitus, chronic pain, ADHD, epilepsy, suboptimal memory function, and suboptimal sleep patterns are known in the art. Analogously, neuronavigated transcranial systems and methods for applying radiation to target brain regions of an individual to modify the permeability of the blood-brain barrier for drug delivery, or of a patient for treating brain tumors (including meningiomas, acoustic neuroma's pituitary, tumors, gliomas, metastatic brain tumors), arteriovenous malformations, trigeminal neuralgia, epilepsy, Parkinson's disease, essential tremor, pain syndromes, and cavernous malformations are known in the art. Finally, neuronavigated transcranial systems and methods for sensing brain energy emission from target brain regions of an individual for functional localization of brain functions before surgery for tumors or epilepsy, diagnosing epilepsy and identifying seizure types, localizing brain areas responsible for specific cognitive functions, assessing cerebral hemodynamics in conditions like stroke or traumatic brain injury, monitoring cerebral hemodynamic responses during therapeutic interventions for stroke rehabilitation, helping diagnose sleep disorders, and monitoring cerebral oxygenation in premature infants to prevent brain damage are known in the art.

For each of these disorders or diseases, a distinct set of brain regions is known to be functioning abnormally or anatomically abnormal, and one or more of these regions must be located and accurately targeted during stimulation, radiation, or sensation for successful treatment or diagnosis. In order to reliably stimulate a desired brain region, an energy delivery and/or detection instrument needs to be consistently and accurately placed at a target scalp location overlying that brain region, and must remain at that site throughout the entire energy delivery and/or detection procedure. Stimulation of off-target brain regions may reduce or eliminate the efficacy of the treatment, and in the worst case scenario may lead to worsening of symptoms, excessive pain during treatment, or rarely, a serious adverse event such as a seizure. Radiation of off-target brain regions can lead to neurological deficits, brain swelling, radiation necrosis, secondary tumors, cognitive deterioration, endocrine dysfunction, and vascular damage. Incorrectly placing a brain energy emission detection device (e.g. EEG, MEG) can lead to missed diagnosis, delayed treatment, inaccurate localization of a tumor to be operated on, or reduce signal quality.

The process of positioning the brain energy delivery and/or detection instrument on the head, and maintaining it in place during treatment, is known as ‘neuronavigation.’ In current clinical practice, the most common approach to neuronavigation is to place a fabric cap on a patient's head, perform measurements of the head and scalp, and use these measurements to define a coordinate system (e.g., the ‘10-20 international encephalography system’) which provides markers on the patient's head. A device operator would then use these marks to specify the target site on the cap, place the brain energy delivery and/or detection instrument over the marked target site, and then trace a (partial) outline of the instrument on the cap for use in maintaining consistent instrument orientation during treatment sessions, and from session to session. However, this method may be imprecise, and does not provide direct visual confirmation that the center of the instrument is (1) directly over the target location, or (2) actually in physical contact with (i.e., touching) the patient's head.

To non-invasively & precisely stimulate, sense, or radiate the brain a neurotechnology needs to be in the correct location on the head. For some technologies like transcranial electrical stimulation (TES) or electroencephalography (EEG), the stimulation of the brain or detected signal in sensing from the brain is fairly uniform over millimeters or even centimeters of the scalp. In this case, the positioning does not need to be that precise, but signal quality is still improved if it is. However, there are many technologies—like Transcranial magnetic stimulation (TMS), magnetoencephalography (MEG), or gamma knife radiation—each of which has an effective field of stimulation, sensation, or radiation which is relatively small. Thus, they each require technology to assist in their placement. Unfortunately such neuronavigation technology—like frameless stereotaxy using infrared or electromagnetic fields—is complicated, bulky, and expensive. We have developed a unique system that allows for the same neuronavigation precision, but instead using direct visualization by means of an imaging device placed directly above the focus of stimulation, sensation, or radiation.

In addition, patients may move during treatment and/or navigational aids may slip out of the desired position. If this occurs, the technician operating the device must pause treatment, readjust position of the instrument, and then resume the procedure. At worst, a patient can move his or her head in such a way that the brain energy delivery and/or detection instrument moves but is not observably positioned off target such that the session continues, with potential adverse consequences as identified above. Present methods lack a direct visual or other record confirming that (1) the brain energy delivery and/or detection instrument is optimally located over the desired target area, (2) the brain energy delivery and/or detection instrument remains over the desired target area throughout the stimulation session, and that (3) the brain energy delivery and/or detection instrument remains in physical contact with the surface of the patient's head during the entire session.

A more complex, less commonly used approach to neuronavigation (FIG. 1) involves a computerized frameless stereotaxic positioning system comprised of: (1) a set of optical position markers such as small reflective beads 70 attached in a specific 3-dimensional configuration to the TMS coil as well as to a tracker on the patient's head, (2) a stereo camera 71 that visualizes and localizes the markers in 3-dimensional space, and (3) computer software 72 which uses 3-dimensional marker position information from the camera to impute the relative positions and orientations of the patient's head and the coil, and then provides a visualization of these positions on a screen viewed by the operator as a neuronavigation guide before and during treatment.

Such a system usually, but not always, also includes (4) an MRI or other 3-dimensional image of the patient's head and brain, which the software aligns with the 3-dimensional imputed head position, so that the operator may visualize the brain region at the focus of the coil in real-time during coil positioning and treatment.

As examples, JP 2003-180649A and JP 2004-000636A disclose techniques for TMS coil neuronavigation using, for example, an optical tracking system employing infrared reflectors, as described above. This technology is commercially available and is also used in clinical settings such as neurosurgical procedures requiring neuronavigation.

JP 2006-320425A discloses another apparatus for positioning a TMS coil against the patient's head by using a multi jointed robot. This approach likewise has several major disadvantages, including the necessity of an MRI scan for every patient, the excessive additional expense and complexity of the apparatus itself, and the need for an operator to undergo an extended training period of several additional weeks to achieve proficiency in accurate use of the system. Further, the system can fail if: (1) the specified target is mistaken, (2) the markers on the coil are incorrectly calibrated, (3) following calibration, the markers on the patient's head move out of position during the session, (4) the operator is insufficiently skilled, or if (5) the coil is not quite in contact with the scalp despite appearing to be so on the neuronavigation system.

The complexity of this external tracking approach with MRI-guidance also greatly reduces the variety, and hence numerosity, of locations where patients may receive treatment or diagnosis by brain energy delivery and/or detection systems. This system is typically confined to a hospital setting because it requires high-field MRIs, as well as significant computing resources, specialized analysts to process the images, and technicians trained to competently operate the cumbersome neuronavigation suite. As a result, this approach is rarely used in the most accessible health care settings such as primary care clinics, mental health centers, assisted living facilities, outpatient specialty clinics, or workplace health centers. Instead, a patient seeking MRI-guided neuronavigated treatment or diagnosis is generally obliged to repeatedly travel to an academic or tertiary health care setting, raising additional barriers of cost and convenience and curtailing the accessibility of TMS treatment for those who need it.

The foregoing discussion of the prior art derives in part from U.S. Pat. No. 10,004,915 (the '915 Patent) wherein there is described a TMS system comprising a TMS alignment system comprising a means for generating magnetic field, the magnetic field generating means having a coil for generating a variable magnetic field to be applied to a certain part of patient's head and a holder for holding the coil; and a camera means for recognizing a predetermined reference marking made on a specific portion of the ear of the patient, (e.g., the tragus); the magnetic field generating means and the recognizing means being designed so that an alignment of the recognizing means with the marking causes the coil to be set in a proper posture with respect to the certain part of the patient's head.

According to the '915 Patent, with the aforesaid arrangement, the magnetic field generating means can be positioned with respect to the reference marking of a specific portion of the patient's ear, allowing the user of the TMS system to position the magnetic field generating means without skill which is needed for conventional systems.

The recognition means of the '915 Patent includes at least one imaging device, i.e., cameras carried on external arms extending from an apparatus. Alignment includes aligning an optical axis of the imaging device with the marking. This allows that the coil is positioned in the proper posture with respect to the specific part of the patient.

Preferably, the TMS system of the '915 Patent further comprises an optical device capable of emitting a directional beam, the optical device being provided adjacent the imaging device, wherein the alignment includes aligning an intersection of the optical axis of the optical device with the marking. This allows that the TMS coil is positioned in the proper position with respect to the specific part of the patient.

In another embodiment of the '915 Patent, the TMS system further comprises a moving mechanism for moving the coil holder on and along a surface of the patient's head; and a controlling means for controlling the moving mechanism in accordance with an output from the recognition means to automatically position the holder with respect to the marking.

As noted earlier, a problem with the TMS alignment system proposed in the '915 Patent is that the system requires marking directly on the patient, the system is bulky, and the arms required for holding the cameras or imaging devices are bulky and themselves prone to bending and/or misalignment. The alignment markings on the patient can also be obscured by the patient's hair. The additional components also bear the risk of inaccurately imputing the coil's actual position, as explained above. Lastly, the additional components reduce the overall accessibility of TMS treatment, by requiring technicians to undergo extensive additional training in order to operate the neuronavigation apparatus correctly, and by limiting treatment location to areas where a stationary multi-ton MRI scanner happens to reside.

### SUMMARY OF THE DISCLOSURE

The present disclosure is based on the premise that major sources of potential error and uncertainty of treatment can be removed from the process of brain energy delivery and/or detection instrument neuronavigation if: (1) the target site on the head can continuously be precisely visualized, and (2) a contact sensor can directly indicate whether the center of the brain energy delivery and/or detection instrument is in contact with the scalp during the entire procedure. In the present disclosure, we provide an optical scalp-landmarking approach which allows for much higher consistency in positioning a brain energy delivery and/or detection instrument over a given site on the scalp from session to session, as well as providing a direct visual record (as opposed to an imputed calculation) of whether the instrument was properly positioned and maintained in this position and in contact with the scalp consistently throughout each session of stimulation. Said another way: rather than using externally-placed sensors and markers on the head to infer TMS coil location from an external perspective (as in FIG. 1), our approach uses sensors placed on the instrument itself to, in essence, provide any brain energy delivery and/or detection instrument's perspective. Moreover, since our novel approach allows the technician and supervising physician to directly visualize the target site while placing the instrument, the time required to train a new technician to proficiency is greatly reduced, rather than extended. Moreover, a verifiable record of placement accuracy may be generated during each treatment session. Finally, our approach does not necessitate the use of costly and cumbersome additional components such as stereo cameras, MRI machines and intensive processing software, or reflector markers requiring calibration prior to treatment. This reduction in cost and complexity, as well as the marked acceleration of an operator's learning curve to proficiency, facilitates more widespread access to neuronavigated treatment and diagnosis in a broader range of settings-outside the more limited number of specialized centers which host large, expensive apparatus, requiring extensive personnel.

Broadly, in accordance with Aspect A of the present disclosure, we provide a neuronavigated transcranial brain energy delivery and/or detection system comprising: i) a brain energy delivery device including an energy delivery instrument configured to be placed over a target brain region of an individual's head for energy delivery; and ii) a power generation unit for energizing the energy delivery instrument, if the brain energy delivery device requires power; and/or iii) a brain energy detection device including an energy detection instrument configured to be placed over a target brain region of an individual's head for energy detection; iv) a receiver unit for receiving the signal from the energy detection instrument, if the brain energy detection device requires a receiver; and v) one or more imaging devices incorporated with the energy delivery and/or detection instrument and configured to permit direct visualization of the position of the energy delivery and/or detection instrument on an individual's head, over the target brain region.

According to one embodiment of Aspect A, the imaging device comprises one or more cameras, selected from the group consisting of one or more visible light imaging cameras, one or more ultraviolet light imaging cameras or one or more infrared imaging cameras.

In accordance with another embodiment of Aspect A, the one or more imaging devices comprise a single camera located centrally relative to the energy delivery and/or detection instrument, two or more cameras located to the sides of the energy delivery and/or detection instrument, or two or more cameras located away from the center but within a housing of the energy delivery and/or detection instrument.

In a further embodiment of Aspect, the brain energy delivery and/or detection system further comprises one or more accelerometers configured to sense orientation placement and/or changes in orientation of the energy delivery and/or detection instrument, and/or one or more contact sensors configured to detect contact and force between the energy delivery and/or detection instrument and the individual's head, wherein the contact sensor comprises one or more force-sensitive resistors, one or more capacitive touch sensors, or one or more ultrasonic position/touch sensors. wherein the neuronavigated brain energy delivery and detection system optionally is configured to regulate energy delivery to the brain in the event the one or more imaging devices or one detection instruments and/or more sensors detects movement of the neuronavigated transcranial brain energy delivery and detection systems away from the target brain region.

In a further embodiment of Aspect A, the neuronavigated transcranial brain energy delivery and/or detection system further comprises one or more imaging devices external to the energy delivery and/or detection instrument, and configured to permit simultaneous visualization of the individual's head as well as the energy delivery and/or detection instrument, and wherein the imaging device comprises one or more cameras, one or more LIDAR detectors, or one or more ultrasonic detectors.

In a still further embodiment of Aspect A, the neuronavigated transcranial brain energy delivery and/or detection system further comprises a memory device configured to create a record of the energy delivery and/or detection instrument's imaging devices, contact sensors, accelerometers, or other sensors before, during, and/or after use, which may be used to infer the instrument's position relative to the individual's head.

In yet another embodiment of Aspect A, the imaging device is configured to transmit an image of the individual's scalp vasculature, the individual's skin patterns, the individual's skull bone structure, or the individual's brain tissue.

In another embodiment of Aspect A, the brain energy delivery and/or detection system is selected from the group consisting of a brain stimulation system comprising: i) transcranial photo biomodulation using infrared or coherent light; ii) transcranial focused ultrasound; iii) transcranial magnetic stimulation; iv) transcranial electrical stimulation; or a brain irradiation system comprising: v) particle-beam, including gamma particles and electrons; or vi) photons from the electromagnetic spectrum including radio waves, x-rays, ultraviolet and gamma rays or a brain activity sensing system comprising: vii) an optical imaging system selected from near infrared spectroscopy (NIRS), diffuse optical tomography (DOT), or optical coherence tomography (OCT), Laser speckle imaging (LSI), Photoacoustic imaging (PAI), and Laser Doppler imaging (LDI); viii) transcranial focused ultrasound (FUS); or ix) magnetoencephalography (MEG); or a brain radiation detection system comprising: x) particle-detector, including gamma particles and electrons; or xi) photons from the electromagnetic spectrum including radio waves, x-rays, ultraviolet and gamma rays.

In still another embodiment of Aspect A, the neuronavigated transcranial brain energy delivery and/or detection system further comprises a support arm configured for supporting the energy delivery and/or detection instrument, wherein the support arm comprises one or more elongated rods having one or a plurality of sections filled at least in part with an electrorheological fluid configured to reversibly change viscosity in response to an applied electromagnetic field.

According to Aspect B, the present disclosure also provides a neuronavigated transcranial kit comprising the neuronavigated transcranial brain energy delivery and/or detection system as above described with reference to Aspect A, and a head cap having indicia, including target markings, text, visual texture, images, tessellations, grids, symbols, visual codes, and/or color markings configured to overlie anatomical locations on the head of the individual.

In one embodiment of Aspect B, the transcranial magnetic stimulation kit includes cap shape features configured to overlie target areas of the head of the individual, and/or including indicia configured to permit continuous measurement of the relative position and orientation of the cap versus the individual's head before, during, and after use.

In another embodiment of Aspect B, the neuronavigated energy delivery and/or detection kit further comprises a power/receiver cable, detachable from the energy delivery and/or detection instrument, and configured to run between the power generator and the brain energy delivery and/or brain energy detection device.

According to Aspect C of the disclosure, there is provided a method for delivering and/or detecting energy from a target brain region of an individual by directing energy onto or receiving energy from the target brain region, which method comprises: i) providing a neuronavigated transcranial brain energy delivery and/or detection system including an energy delivery and/or detection instrument and one or more imaging devices as above described with reference to Aspect A, ii) positioning the energy delivery and/or detection instrument over the target region using the imaging devices to visualize the placement of the energy delivery and/or detection instrument in terms of the three translational or rotational dimensions with respect to the target region, wherein correct positioning of the energy delivery and/or detection instrument is optionally prompted by visual, auditory and/or haptic feedback; and iii) activating and deactivating the one or more energy delivery and/or detection instruments according to a usage protocol.

In one embodiment, Aspect C includes the step of providing an individual with a head cap having indicia, including target markings, text, visual texture, images, tessellations, grids, symbols, visual codes, and/or color markings configured to overlie anatomical locations on the head of the individual; and positioning the energy delivery and/or detection instrument over the target region using the imaging devices to visualize placement of the energy delivery and/or detection instrument relative to the indicia.

In another embodiment of Aspect C, correct positioning of the energy delivery and/or detection instrument is prompted by visual, auditory and/or haptic feedback.

In yet another embodiment of Aspect C, the treatment comprises a treatment selected from the group consisting of a brain stimulation system comprising: i) transcranial photo biomodulation using infrared or coherent light; ii) transcranial focused ultrasound; iii) transcranial magnetic stimulation; iv) transcranial electrical stimulation; or a brain irradiation system comprising: v) particle-beam, including gamma particles and electrons; or vii) photons from the electromagnetic spectrum including radio waves, x-rays, ultraviolet and gamma rays or a brain activity sensing system comprising: vii) an optical imaging system selected from near infrared spectroscopy (NIRS), diffuse optical tomography (DOT), or optical coherence tomography (OCT), Laser speckle imaging (LSI), Photoacoustic imaging (PAI), and Laser Doppler imaging (LDI); viii) transcranial focused ultrasound (FUS); or ix) magnetoencephalography (MEG); or a brain radiation detection system comprising: x) particle-detector, including gamma particles and electrons; or xii) photons from the electromagnetic spectrum including radio waves, x-rays, ultraviolet and gamma rays.

In accordance with Aspect D of the disclosure there is provided a transcranial magnetic stimulation (TMS) coil head configured to be placed over a target brain region for treatment, wherein the TMS coil head comprises a housing containing one or more coil windings within the housing, and a phase change material (PCM) in contact with one or more windings within the housing, optionally with non-contiguous winding paths to maximize the PCM's contact surface area with the windings or wherein the TMS coil head comprises a conductive cooling unit in physical contact with the TMS coil head, preferably the patient-facing surface, to dissipate heat, wherein the cooling unit functions either as a passive heatsink or actively cools using conduction, convection, or electrical cooling, preferably as a Peltier Thermocouple.

In accordance with Aspect E of the disclosure, there is provided a brain energy delivery or brain energy detection instrument comprising one or more imaging devices incorporated into the energy delivery and/or detection instrument and configured to permit direct visualization of a position of the energy delivery and/or detection instrument, and a permanent or removable mounting joint, on a top of the energy delivery and/or detection instrument, laterally centered on a central vertical axis through the energy delivery and/or detection instrument, wherein the energy delivery and/or detection instruments optionally includes a complementary part configured to connect to the mounting joint via a single action quick release mechanism.

In accordance with Aspect F of the disclosure, there is provided a method for stimulating a target brain region by transcranial magnetic stimulation, which method comprises: i) providing a neuronavigated transcranial magnetic stimulation system including a TMS coil and one or more imaging devices according to Aspect A above described; ii) positioning the TMS coil over the target region using the imaging devices to visualize placement of the TMS coil; and iii) activating and/or deactivating the one or more magnetic induction coils according to a treatment protocol.

In accordance with one embodiment of Aspect F, the method includes the step of providing a patient with a head cap having indicia markings in the form of a grid, text and/or color markings configured to overlie anatomical locations on the head of the patient; and positioning the TMS coil over the target region using the imaging devices to visualize placement of the TMS coil relative to the indicia, wherein the correction positioning of the TMS coil optionally is prompted by visual, auditory and/or haptic feedback.

More particularly, in accordance with a preferred embodiment of the present disclosure, there is provided a neuronavigated transcranial brain energy delivery and/or detection system comprising: i) a brain energy delivery device including an energy delivery instrument configured to be placed over a target brain region of an individual's head for energy delivery; and ii) a power generation unit for energizing the energy delivery instrument, if the brain energy delivery device requires power; and/or iii) a brain energy detection device including an energy detection instrument configured to be placed over a target brain region of an individual's head for energy detection; and iv) a receiver unit for receiving the signal from the energy detection instrument, if the brain energy detection device requires a receiver; and v) one or more imaging devices incorporated with the energy delivery and/or detection instrument and configured to permit direct visualization of the position of the energy delivery and/or detection instrument on an individual's head, over the target brain region.

In one embodiment of the neuronavigated transcranial brain energy delivery and/or detection system, the imaging device comprises one or more cameras, preferably one or more visible light imaging cameras, one or more ultraviolet light imaging cameras or one or more infrared imaging cameras.

In one embodiment the imaging device preferably comprises a single camera located centrally relative to the brain energy delivery and/or detection instrument, and optionally two or more cameras located to the sides of the instrument, or two or more cameras located away from the center but within a housing of the instrument.

In another embodiment, the neuronavigated transcranial brain energy delivery and/or detection system further comprises one or more accelerometers configured to sense orientation placement and/or changes in orientation of the instrument, and/or one or more contact sensors configured to detect contact and force between the instrument and the patient's head, wherein the contact sensor preferably comprises one or more force-sensitive resistors, one or more capacitive touch sensors, or one or more ultrasonic position/touch sensors, and optionally wherein the neuronavigated brain energy delivery and detection system is configured to regulate energy delivery and/or detection based on the instrument's position, velocity, or acceleration in any of the three translational or rotational dimensions, as inferred from measurements from embedded imaging devices or sensors, and relative to the target brain region.

In another embodiment, the neuronavigated transcranial brain energy delivery and/or detection system further comprises one or more imaging devices external to the instrument, and configured to permit simultaneous visualization of the patient's head as well as the instrument, wherein the imaging device preferably comprises one or more cameras, one or more LIDAR detectors, or one or more ultrasonic detectors.

In yet another embodiment, the neuronavigated transcranial brain energy delivery and/or detection system, further comprises a memory device configured to create a record of the instrument position before, during, and/or after the procedure.

In a further embodiment of a neuronavigated transcranial brain energy delivery and/or detection system, the imaging device is configured to transmit an image of the patient's scalp vasculature, the patient's skin patterns, the patient's skull bone structure, or the patient's brain tissue, as the case may be.

In yet another embodiment of a neuronavigated transcranial brain energy delivery and/or detection system the system is selected from the group consisting of a neurostimulation system comprising:
i. transcranial photo biomodulation using infrared or coherent light;
ii. transcranial focused ultrasound;
iii. transcranial magnetic stimulation;
iv. transcranial electrical stimulation; or a neuronavigated neuroimaging system comprising:
v. an optical imaging system selected from near infrared spectroscopy (NIRS), diffuse optical tomography (DOT), or optical coherence tomography (OCT), Laser speckle imaging (LSI), Photoacoustic imaging (PAI), and Laser Doppler imaging (LDI);
vi. transcranial focused ultrasound (FUS); or
vii. magnetoencephalography (MEG); or a neuronavigated neuroradiation system comprising:
viii. particle-beam, including gamma particles and electrons; or
ix. photons from the electromagnetic spectrum including radio waves, x-rays, ultraviolet and gamma rays.

In a further embodiment, the neuronavigated transcranial brain energy delivery and/or detection system further comprises a support arm configured for supporting the treatment instrument, wherein the support arm comprises one or more elongated rods having one or a plurality of sections of varied rigidity, optionally containing—at least in part—an electrorheological, magnetorheological, pneumatic, or hydraulic substance configured to reversibly change rigidity in response to an electromagnetic field or by modulating the substance's quantity within the rod.

The present disclosure also provides a neuronavigated transcranial treatment kit comprising the neuronavigated transcranial brain energy delivery and/or detection system as above described, and a head cap having indicia—including target markings, text, visual texture, images, tessellations, grids, symbols, visual codes, and/or color markings—configured to overlie anatomical locations on the head of the individual optionally wherein the cap's geometrical features and/or specific indicia are configured to align with or emphasize distinct anatomical features of the head of the individual, allowing for continuous measurement of the cap's relative position and orientation versus the individual's head before, during, and after use. The neuronavigated energy delivery and/or detection kit further comprises a power/receiver cable, detachable from the energy delivery and/or detection instruments and configured to run between the power generator or signal receiver, and the brain energy delivery and/or brain energy detection device.

In one embodiment of the transcranial magnetic stimulation kit we include markings configured to overlie target areas of the head of the patient, and/or including markings configured to permit continuous measurement of the relative position and orientation of the cap versus the patient's head before, during, and/or after treatment.

The present disclosure also provides method for treating a target brain region of a patient in need of treatment by directing energy onto or receiving energy from the target brain region, which method comprises: i) providing a neuronavigated transcranial brain energy delivery and/or detection system including an energy delivery and/or detection instrument and one or more imaging devices as above described; ii) positioning the energy delivery and/or detection instrument over the target region using the imaging devices to visualize placement of the energy delivery and/or detection instrument in terms of the three translational or rotational dimensions with respect to the target region, wherein correct positioning of the energy delivery and/or detection instrument is optionally prompted by visual, auditory and/or haptic feedback; and iii) activating and deactivating the one or more energy delivery and/or detection instruments according to a treatment protocol.

In one embodiment the method includes the step of providing a patient with a head cap having indicia including target markings, text, visual texture, images, tessellations, grids, symbols, visual codes, and/or color markings—configured to overlie anatomical locations on the head of the individual; and positioning the energy delivery and/or detection instrument over the target region using the imaging devices to visualize placement of the energy delivery and/or detection instrument relative to the indicia.

In still another embodiment of the method the treatment comprises a treatment selected from the groups consisting of a neurostimulation system comprising:
i. transcranial photo biomodulation using infrared or coherent light;
ii. transcranial focused ultrasound;
iii. transcranial magnetic stimulation;
iv. transcranial electrical stimulation; or a neuronavigated neuroimaging system comprising:
v. an optical imaging system selected from near infrared spectroscopy (NIRS), diffuse optical tomography (DOT), or optical coherence tomography (OCT), Laser speckle imaging (LSI), Photoacoustic imaging (PAI), and Laser Doppler imaging (LDI);
vi. transcranial focused ultrasound (FUS); or
vii. magnetoencephalography (MEG); or a neuronavigated neuroradiation system comprising:
viii. particle-beam including gamma particles and electrons; or
ix. photons from the electromagnetic spectrum including radio waves, x-rays, ultraviolet and gamma rays.

The present disclosure also provides a transcranial magnetic stimulation (TMS) system configured to be placed over a target brain region for treatment, wherein the TMS coil head comprises a housing containing one or more coil windings within the housing, and a phase change material (PCM) in contact with one or more windings within the housing, optionally with non-contiguous winding paths to maximize the PCM's contact surface area with the windings, or wherein the TMS coil head comprises a conductive cooling unit in physical contact with the TMS coil head, preferably the patient-facing surface, to dissipate heat, wherein the cooling unit functions either as a passive heatsink or actively cools using conduction, convection, or electrical cooling, preferably as a Peltier Thermocouple.

The present disclosure also provides a brain energy delivery or brain energy detection instrument comprising one or more imaging devices incorporated into the energy delivery and/or detection instrument and configured to permit direct visualization of a position of the energy delivery and/or detection instrument, and a permanent or removable mounting joint, on a top of the energy delivery and/or detection instrument, laterally centered on a central vertical axis through the energy delivery and/or detection instrument, wherein the energy delivery and/or detection instruments optionally includes a complementary part configured to connect to the mounting joint via a single action quick release mechanism.

The present disclosure also provides a method for stimulating a target brain region by transcranial magnetic stimulation, which method comprises: i) providing a neuronavigated transcranial magnetic stimulation system including a TMS coil and one or more imaging devices as above described; ii) positioning the TMS coil over the target region using the imaging devices to visualize placement of the TMS coil; and iii) activating and/or deactivating the one or more magnetic induction coils according to a treatment protocol, and optionally including the step of providing a patient with a head cap having indicia—including target markings, text, visual texture, images, tessellations, grids, symbols, visual codes, and/or color markings—configured to overlie anatomical locations on the head of the patient; and positioning the TMS coil over the target region using the imaging devices to visualize placement of the TMS coil relative to the indicia, wherein the correction positioning of the TMS coil optionally is prompted by visual, auditory and/or haptic feedback.

More particularly, as applied to a TMS treatment system, we incorporate one or more imaging devices into a TMS coil so as to permit direct visualization of the placement of the center of the TMS coil on the head. In one embodiment, we incorporate a single camera directly in the center of the TMS coil so as to permit direct visualization of the area under a TMS coil. In another embodiment of the disclosure, we incorporate two or more cameras placed off-center and/or on the sides of the TMS coil. The camera(s) may comprise visual light imaging capabilities, ultraviolet light imaging capabilities or infrared light imaging capabilities.

In another embodiment, we also incorporate into the TMS coil one or more contact sensors so as to detect whether the patient's head is in contact with the coil before, during, and until the treatment session concludes. The contact sensors may comprise force-sensitive resistors, capacitive touch sensors, ultrasonic position/touch sensors, and/or thermal/infrared sensors.

In another embodiment, we also incorporate one or more imaging devices external to the coil, configured to allow for simultaneous visualization of the patient's head (and any associated markings) as well as the coil, as an independent measure of their relative positions. These additional coil-external cameras may comprise one or more cameras, LIDAR detectors, and/or ultrasonic detectors.

In one embodiment, we provide a specialized treatment cap having indicia with various markings including grid markings, text and/or color markings corresponding to specific anatomical locations on the head of the patient.

In another embodiment, the TMS system is configured to record and optionally, transmit, in real time, a video of the TMS coil placement during treatment. Also, in yet another embodiment, we include one or more accelerometers in the TMS coil so as to provide a supplementary record of the orientation of the TMS coil throughout treatment, so that the provider can detect any subtle drift or deviation of the coil during treatment, and make adjustments to the TMS coil orientation accordingly.

More particularly, in one aspect we provide a transcranial magnetic stimulation system comprising: a TMS system configured to generate a magnetic field to be applied to a patient's brain region, the TMS system comprising a TMS pulse generator as well as an inductor coil; and one or more imaging devices incorporated into the coil and configured to permit direct visualization of the TMS coil on the patient's head. The imaging device may comprise one or more cameras, preferably one or more visible light imaging cameras, one or more ultraviolet light imaging cameras, or one or more infrared imaging cameras.

The transcranial magnetic stimulation system may further comprise one or more accelerometers configured to sense orientation placement and/or changes in orientation of the TMS coil.

The transcranial magnetic stimulation system also may further comprise a memory device configured to create a video record of TMS coil placement during treatment.

We also provide a transcranial magnetic stimulation neuronavigation kit comprising the transcranial magnetic stimulation system as above described, and patient head cap having grid markings, text and/or color markings configured to overlie anatomical locations on the head of the patient. The patient head cap may include markings configured to overlie target areas of the head of the patient, and/or markings configured to permit continuous measurement of the position and orientation of the cap relative to the patient's head before, during, and after treatment.

In yet another embodiment, we provide a specialized treatment cap having indicia with various markings including grid markings, text and/or color markings corresponding to specific anatomical locations on the head of the patient.

In yet another embodiment, rather than employing the current standard of care of placing the treatment cap a few centimeters above the eyebrows, measuring the distance from nasion to cap brim, and then for every subsequent session, trying to place the cap back exactly that same distance, remeasuring each time, we provide a cap geometry where the brim comes to a point on the midline. This point can be immediately visually confirmed to be correctly placed or not without the need for measuring tape. Additionally, we place indicia on the cap that denotes where the cap should be with respect to the tragus (the point flap of skin on the ear), as an additional marker to ensure reliable fit of the cap on the head.

With both the point front, and tragus markers on both sides, these three markers can be used not only for basic visual confirmation, but we can employ AI algorithm to ensure proper cap positioning, by using a smartphone camera and slowly wave it around the patient from left to front to right side to ensure the cap is properly positioned.

In another embodiment, the TMS system is configured to record and optionally transmit, in real time, a video of the TMS coil placement during treatment. Also, in yet another embodiment, we include one or more accelerometers in the TMS coil configured to provide a supplementary record of the orientation of the TMS coil throughout treatment, so that the provider can detect any subtle drift or deviation of the coil during treatment and make adjustments to the TMS coil orientation accordingly.

In another embodiment we provide a transcranial magnetic stimulation system comprising: a TMS system configured to generate a magnetic field to be applied to a patient's brain region, the TMS system comprising a TMS pulse generator as well as an inductor coil; and one or more imaging devices incorporated into the coil and configured to permit direct visualization of the TMS coil on the patient's head. The imaging device may comprise one or more cameras, preferably one or more visible light imaging cameras, one or more ultraviolet light imaging cameras, or one or more infrared imaging cameras.

The transcranial magnetic stimulation system may further comprise one or more accelerometers configured to sense orientation placement and/or changes in orientation of the TMS coil.

The transcranial magnetic stimulation system also may further comprise a memory device configured to create a video record of TMS coil placement during treatment.

We also provide a transcranial magnetic stimulation neuronavigation kit, comprising the transcranial magnetic stimulation system as above described, and patient head cap having grid markings, text and/or color markings configured to overlie anatomical locations on the head of the patient. The patient head cap may include markings configured to overlie target areas of the head of the patient, and/or markings configured to permit continuous measurement of the position and orientation of the cap relative to the patient's head before, during, and after treatment.

A feature and advantage of the transcranial magnetic stimulation system of our disclosure is the provision of an imaging device or camera central to a center of the TMS coil head configuration, which permits direct visualization of the TMS coil(s) on the patient's head.

Conventional or so-called “donut” coils TMS coil head 102A, 102B employed in prior art TMS coil heads as illustrated in FIGS. 10 and 11 are geometrically unsuited for incorporation of an imaging device or camera centrally located between the coils.

In accordance with the present disclosure, we provide TMS coil configuration that permits the positioning of one or more imaging devices, i.e., one or more cameras including a single camera central to the center of the TMS coil head. However, in order to facilitate positioning a camera central to the center of the TMS coil head, we cannot simply wrap or tie our coils together as in the case of prior art traditional “donut” coils 102A, 102B using string 104 or tape 106 to bind the wires together (FIG. 10). Rather, in accordance with the present disclosure we provide the inside of the bottom and/or top of the coil head holder with a plurality of spacers or pegs for holding the wires in place.

Conventional prior art transcranial magnetic stimulation systems also rely on circulating a coolant through the TMS coil head to dissipate excess heat and keep the TMS coil head at a proper and comfortable temperature for the patient. However, providing our novel wire coil geometry that permits us to locate an imaging device including a camera central to the center of the TMS coil head prevents cooling the TMS coil head by continuously circulating a cooling fluid through the coils. Accordingly, in accordance with the present disclosure we employ a phase change material (PCM) permanently packed around the wires in our TMS coil head. The PCM has sufficient thermal energy absorption capacity to cool the coils for the duration of a treatment. Accordingly, in accordance with further aspect of the disclosure, the TMS coil head is configured to be readily swapped out between treatment sessions. To facilitate this, in accordance with the present disclosure, we provide a detachable cable configured to be detachable fixed to the TMS coil head or to the TMS pulse generator. Traditional TMS coils circulate cooling fluid through a cooler so that they can be used non-stop. In accordance with the present disclosure, each TMS system is provided with multiple TMS coils so that the health care provider readily can swap out coil heads between patients. Thus, as distinguished with conventional prior art TMS coil heads that are configured to circulate coolant to the TMS coil heads and also energize the coil(s), our TMS coil head is far simpler in construction and does not need to be configured to circulate a coolant. Also, with our PCM cooled coil heads, the cable needs only a single junction, i.e., to plug into a TMS pulse generator.

In another aspect, with our PCM cooled coil heads, we provide a detachable power cable that can also detach from the TMS coil head, configured to deliver electrical power from a pulse generator to the TMS coil head. This is another advantage over conventional TMS coil heads, which require fixed and expensive cabling to both circulate coolant and deliver electrical pulses. Employing a PCM as a coolant sealed in the TMS coil head provides additional advantages. For one, the density of PCM is much lighter than traditional liquid coolants such as Galden® HT 135. As a result, our TMS coil head is lighter in weight which means we do not require TMS coil holder arms which are heavy, bulky and difficult to use (see FIG. 11).

Additionally, because our TMS coil heads are lighter in weight, we can use light weight compact ergonomically shaped handles on the TMS coil heads on the top of the coil head, as opposed to traditional TMS coil heads as illustrated in FIG. 11 that have a “paddle” handle 110 extending out from a side of the coil head 112, which forces the operator to handle the coil head like a sword which is tiring for the operator.

In another aspect, the PCM further facilitates thermal dispersions by including thermally conductive materials such as metal fines, e.g., copper, tin or aluminum, carbon allotropes such as graphite or graphene, or a thermal paste.

Our TMS coil head shape with a compact ergonomically shaped handle on the top of the coil head also permits us to mount the power cable through the top of the coil head. This provides us with another advantage over conventional paddle shaped coil heads in which the power hoses are connected through the handle.

In one aspect, the TMS coil head further comprises a permanent or removable mounting joint on a top of the coil head, vertically centered on a central vertical axis of the coil winding.

In another aspect, a complementary fixture to which the mount joint connects, has a single action quick release mechanism.

More particularly, one aspect of the disclosure provides a TMS coil head configured to be placed over a target brain region for treatment, wherein the TMS coil head comprises a housing containing one or more coil windings within the housing, and a PCM in contact with the one or more windings within the housing.

In one aspect the TMS coil head includes one or more imaging devices including an imaging device located central to a center of the TMS housing and configured to permit direct imaging of the center of the TMS coil housing on a patient's head.

In one aspect the coil windings are located to either side of the center of the TMS coil head.

In one aspect the TMS coils to either side of the center of the TMS coil head are mirror images of one another.

In another aspect the housing contains a base and a top, and wherein the TMS coils are fixed in position around spacers or pegs extending from the base or the top.

In another aspect the TMS coils are fixed in position around spacers or pegs extending from the base, and held down by pegs extending from the top, or vice versa.

In a further aspect the coils are adhesively held in place in the base or the top.

In another aspect, the TMS coil head further comprises an ergonomically shaped handle affixed to a top of the TMS coil head.

In a further aspect the TMS coil head further includes heatsink elements in contact with the PCM.

The present disclosure also provides a TMS system comprising: (1) a pulse generator; and (2) a TMS coil head including a PCM as above described.

In one aspect the TMS system further comprises one or more imaging devices including a single imaging device located central to a center of the TMS coil head and configured to permit direct imaging of the center of the TMS coil head on the patient's head.

In a further aspect the TMS system includes one or more imaging devices comprising one or more cameras, including a single camera central to the center of the TMS coil head.

In another aspect, the TMS system includes one or more imaging devices comprising one or more visible light imaging cameras, one or more ultraviolet light imaging cameras or one or more infrared imaging cameras.

In another aspect, the TMS system includes one or more imaging devices comprising two or more cameras located to sides of the TMS coil head.

In a further aspect, the TMS system includes one or more imaging devices comprising two or more cameras located away from the center but within a housing of the TMS coil head.

In yet another aspect, the TMS system further comprises one or more accelerometers configured to sense orientation placement or changes in orientation of the TMS coil head.

In a further aspect, the TMS system further comprises one or more contact sensors configured to detect contact and force between the TMS coil head and the patient's head.

In a further aspect, the TMS system includes one or more contact sensors comprising one or more force-sensitive resistors, one or more capacitive touch sensors or one or more ultrasonic position/touch sensors.

In a further aspect, the TMS system further comprises one or more imaging devices external to the TMS coil head and configured to permit simultaneous visualization of the patient's head as well as the TMS coil head.

In a still further aspect, the TMS system includes one or more imaging devices external to the one or more TMS coil head comprising one or more cameras, one or more LIDAR detectors, or one or more ultrasonic detectors.

In a further aspect the TMS system further comprises a memory device configured to create a record of the TMS coil head position before and during treatment.

In another aspect, the TMS system includes one or more imaging devices configured to transmit an image of the patient's scalp vasculature, the patient's skin patterns, the patient's skull bone structure, or the patient's brain tissue, as a case may be.

The present disclosure also provides a treatment cap configured to provide visual guidance for a medical procedure, comprising a skull cap having a pointed midline brim configured to align to the patient's nasion, and/or indicia markings on both sides of the cap configured to align to the tragus of a patient's ears.

The present disclosure also comprises a TMS kit comprising a TMS system, comprising:
i) a pulse generator; ii) a TMS coil head including a PCM as above described, and configured to be placed over a target brain region for treatment; and iii) a patient treatment instrument cap having a brim that comes to a point on the midline, and having indicia markings configured to overlie target locations on the head of the patient.

In one aspect the TMS kit includes a treatment cap having a pointed midline brim configured to align to the patient's nasion, and/or indicia markings including tragus markings on both sides of the cap configured to align with the tragus of the patient's ears, or a point bisecting a line connecting the patient's pupils.

In one aspect the TMS kit further includes a smartphone camera configured to image a position of the head cap.

In a further aspect the neuronavigated TMS kit further comprises one or more imaging devices including a single imaging device central to a center of the TMS coil head and configured to permit direct visualization of the center of the TMS coil head.

In a further aspect the neuronavigated TMS kit further comprises one or more imaging devices including one or more visible light imaging cameras, one or more ultraviolet light imaging cameras or one or more infrared imaging cameras.

In another aspect the neuronavigated TMS kit further comprises one or more contact sensors configured to detect contact and force between the TMS coil head and the patient's head. In a further aspect the neuronavigated TMS kit further comprises one or more imaging devices also including two or more cameras located to sides of the TMS coil head.

In a further aspect the neuronavigated TMS kit further comprises one or more imaging devices also including two or more cameras located away from the center but within a housing of the TMS coil head.

In another aspect the neuronavigated TMS kit further comprises one or more accelerometers configured to sense orientation placement or changes in orientation of the TMS coil head.

In another aspect the neuronavigated TMS kit further comprises one or more contact sensors including one or more force-sensitive resistors, one or more capacitive touch sensors, or one or more ultrasonic position/touch sensors.

In a further aspect the neuronavigated TMS kit further comprises one or more imaging devices external to the TMS coil head and configured to permit simultaneous visualization of the patient's head as well as the TMS coil(s).

In another aspect the neuronavigated TMS kit further comprises one or more imaging devices including one or more cameras, one or more LIDAR detectors, or one or more ultrasonic detectors.

In a further aspect the TMS kit further comprises a memory device configured to create a record of the TMS coil head position before and during treatment.

In another aspect the neuronavigated TMS kit further comprises one or more imaging devices are configured to transmit an image of the patient's scalp vasculature, the patient's skin patterns, the patient's skull bone structure, or the patient's brain tissue, as a case may be.

The present disclosure also provides a method for stimulating a target brain region by TMS, which method comprises: i) providing the neuronavigated TMS system including a TMS coil head containing a PCM; ii) positioning the TMS coil head over the target region using the single imaging device central to the center of the TMS coil head and configured to permit direct visualization and placement of the TMS coil head over the target brain region; iii) activating and deactivating the TMS coil head according to a treatment protocol; and iv) passively cooling the TMS coil head by contact with a contained PCM.

In one aspect the method includes the step of providing the patient with a treatment instrument cap having indicia markings in the form of at least one of a grid, text and color markings configured to overlie anatomical locations on the head of the patient; and positioning the TMS coil head over the target brain region using one or more imaging device to visualize placement of the TMS coil head relative to the indicia. In a particular embodiment the treatment cap has a pointed midline brim configured to align to the patient's nasion, and/or indicia markings on both sides of the cap configured to align with the tragus of the patient's ears.

In another aspect correct positioning of the TMS coil head is prompted by at least one of visual, auditory, and haptic feedback.

TMS pulses are more comfortable for the patient at specific rotations. For example, the TMS coil rotated to 90 degrees might be more comfortable than 0 degrees. However, one problem delivering stimulation at some rotations of conventional paddle shaped coil heads is that the power cable and cooling hoses coming out of the TMS coil head paddle handle may droop down across the patient's face. Patients find this uncomfortable. So rather than position the coil at an annoying position, our system permits us to rotate the coil at 180 degrees relative to the annoying position, and then reverse the polarity of the electricity. Together this still creates the same electric field. Essentially, a normal waveform in one direction is equivalent to the reversed waveform, with the coil upside down.

This rotation-induced flip in polarity is a unique possibility of our system because: 1) we have the only camera+cap with indicia that detects coil rotation and thus allow rotation of the coil head, and 2) our power electronics can effortlessly reverse the waveform. We also can cover the case where the operator may manually decide to flip the waveform polarity if they are just manually using the device.

In yet another embodiment of our disclosure, we provide a TMS coil head configured to be placed over a target brain region for treatment, wherein the TMS coil head comprises a housing containing one or more coil windings within the housing, wherein said one or more coil windings are configured to generate a maximum magnetic field at a common focal point; and one or more imaging devices including a single camera configured to overlie the common focal point so as to permit direct visualization of a position of the TMS coil head relative to a target position on a patient's head.

In one embodiment the TMS coil head comprises two coil windings located to either side of the center of the TMS coil head. In such embodiment the two coil windings preferably are mirror images of one another.

In another embodiment the one or more imaging devices comprises one or more cameras, including the single camera overlying the common focal point of the one or more coil windings. In such embodiment the one or more imaging devices preferably comprises one or more visible light imaging cameras, one or more ultraviolet light imaging cameras or one or more infrared imaging cameras, and/or the one or more imaging devices also comprises two or more cameras located to the sides of the TMS coil head. Alternatively, the one or more imaging devices may also comprise two or more cameras located away from the center but within the housing of the TMS coil head.

In another embodiment, the TMS coil head comprises one or more accelerometers configured to sense orientation placement or changes in orientation of the TMS coil head.

In still another embodiment, the TMS coil head comprises one or more contact sensors configured to detect contact and force between the TMS coil head and the patient's head. In such an embodiment, the contact sensors preferably comprise one or more force-sensitive resistors, one or more capacitive touch sensors or one or more ultrasonic position/touch sensors.

In yet another embodiment, the TMS coil head further comprises one or more imaging devices configured to permit simultaneous visualization of the patient's head as well as the TMS coil head. In such an embodiment, the one or more imaging devices preferably comprises one or more cameras, one or more LIDAR detectors, or one or more ultrasonic detectors.

In another embodiment the TMS coil head further comprises a memory device configured to create a record of the TMS coil head position before and during treatment.

In a preferred embodiment we also provide a contact sensor configured to determine whether the TMS coil remains in the same position and in contact with the scalp during the entire TMS stimulation session. In one embodiment, we provide an optical scalp-landmarking approach which allows for much higher consistency in positioning a TMS coil over a given site on the scalp from session to session, as well as providing a direct visual record (as opposed to an imputed calculation) of whether the coil was properly positioned and maintained in this position and in contact with the scalp consistently throughout each session of stimulation. That is to say, rather than using externally placed sensors and markers on the head to infer the coil location from an external perspective (as in FIG. 1), our approach uses sensors placed on the coil itself to, in essence, provide the coil's perspective. Moreover, since our novel approach allows the technician and supervising physician to directly visualize the target site while placing the coil, the time required to train a new technician to proficiency is greatly reduced, rather than extended. Moreover, a verifiable record of placement accuracy may be generated during each treatment session. Finally, our approach avoids the prior art use of costly and cumbersome additional components such as stereo cameras, MRI machines and intensive processing software, or reflector markers requiring calibration prior to treatment. This reduction in cost and complexity, as well as the marked acceleration of an operator's learning curve to proficiency, facilitates more widespread access to neuronavigated TMS treatment in a broader range of settings outside the more limited number of specialized centers which host large, expensive apparatus, requiring extensive personnel.

In another embodiment of the disclosure, in addition to providing an imaging device configured to provide direct visualization of a coil to locate the focal point of a coil at the target region of the patient's brain, we also incorporate two or more cameras placed off-center and/or on the sides of the TMS coil. The camera(s) may comprise visual light imaging capabilities, ultraviolet light imaging capabilities or infrared light imaging capabilities.

In accordance with another embodiment of our disclosure, we also incorporate into the TMS coil one or more contact sensors configured to detect whether the coil is in contact with the patient's head before, during, and until the treatment session concludes. The contact sensors may comprise force-sensitive resistors, capacitive touch sensors, ultrasonic position/touch sensors, and/or thermal/infrared sensors.

In another embodiment of our disclosure, we also incorporate one or more imaging devices external to the coil, configured to allow for simultaneous visualization of the patient's head (and any associated markings) as well as the coil, as an independent measure of their relative positions. These additional coil-external cameras may comprise one or more cameras, LIDAR detectors, and/or ultrasonic detectors.

In yet another embodiment of our disclosure, we provide a specialized treatment cap having indicia with various markings including grid markings, text and/or color markings corresponding to specific anatomical locations on the head of the patient.

In yet another embodiment of our disclosure, we provide a treatment cap geometry where the brim of the cap comes to a point on the midline. This point can immediately be visually confirmed to be correctly placed or not without the need for measuring tape. Additionally, we place indicia on the cap that denotes where the cap should be with respect to the tragus (the point flap of skin on the ear), as an additional marker to ensure reliable fit of the cap on the head.

With both the point front, and tragus markers on both sides, these three markers can be used not only for basic visual confirmation, but we can employ AI algorithm to ensure proper cap positioning, by using a smartphone camera and slowly wave it around the patient from left to front to right side to ensure the cap is properly positioned.

In yet another embodiment of our disclosure, the TMS system is configured to record and optionally, transmit, in real time, a video of the TMS coil placement during treatment. Also, in still yet another embodiment, we include one or more accelerometers in the TMS coil configured to provide a supplementary record of the orientation of the TMS coil throughout treatment, so that the provider can detect any subtle drift or deviation of the coil during treatment and make adjustments to the TMS coil orientation accordingly.

In another embodiment of our disclosure, we provide a transcranial magnetic stimulation system comprising: a TMS system configured to generate a magnetic field to be applied to a patient's brain region, the TMS system comprising a TMS pulse generator as well as an inductor coil; an imaging device incorporated into the coil and configured to permit direct visualization of the TMS coil to locate the coil on the patient's head so that the focal point of the coil is at the target region of the patient's brain. The imaging device may comprise a camera, preferably a visible light imaging camera, an ultraviolet light imaging camera, or an infrared imaging camera. In another embodiment of our disclosure, the TMS system may further comprise one or more accelerometers configured to sense orientation placement and/or changes in orientation of the TMS coil.

In a further embodiment of our disclosure, the TMS system also may further comprise a memory device configured to create a video record of TMS coil placement during treatment.

We also provide a transcranial magnetic stimulation neuronavigation kit, comprising a TMS system as above described, and a patient head cap having grid markings, text and/or color markings configured to overlie anatomical locations on the head of the patient. The patient head cap may include markings configured to overlie target areas of the head of the patient, and/or markings configured to permit continuous measurement of the position and orientation of the cap relative to the patient's head before, during, and after treatment.

### BRIEF DESCRIPTION OF THE DRAWINGS

Further features of the disclosure will be seen from the following detailed description, taken in conjunction with the accompanying drawings, wherein:

FIG. 1 is a schematic view of a conventional frameless stereotaxic MRI-guided neuronavigation system, in accordance with the prior art;

FIG. 2 is a schematic view of a TMS system in accordance with an embodiment of the present disclosure;

FIG. 3 is a schematic view of a TMS coil in accordance with an embodiment of the present disclosure;

FIG. 4 is a bottom plan view of a TMS coil element of the present disclosure;

FIG. 5 is a block diagram of a power and control circuit of the present disclosure;

FIGS. 6A and 6B are perspective views of a cap element of the present disclosure;

FIGS. 7A-7F are perspective views of alternative cap elements of the present disclosure;

FIGS. 8A-8C are views of a patient's skin, vasculature, bone, and cortical elements of the head;

FIG. 9 is a flow diagram of an embodiment of a method of the present disclosure;

FIG. 10 is a top plan view showing coil windings of a conventional prior art TMS coil head;

FIG. 11 is a perspective view of a conventional prior art TMS system with a conventional prior art TMS coil head;

FIG. 12 is a plan view and FIG. 12A a close-up plan view of a partially disassembled TMS coil head in accordance with an embodiment of the present disclosure;

FIG. 13 is a cross sectional view of a TMS coil head in accordance with an embodiment of the present disclosure;

FIG. 14 is a perspective view and FIG. 14A is a close up view of a TMS coil head in accordance with an embodiment of the present disclosure;

FIG. 14B1 and FIG. 14B2 are side elevational views, in cross section, of a TMS coil head in accordance with the present disclosure;

FIG. 14C is a perspective view of a coil holding arm in accordance with an embodiment of the present disclosure, and FIGS. 14D and 14E are cross-sectional views of a portion of the coil holding arm of FIG. 14C;

FIG. 15 is a cross-sectional view of a detachable power cable in accordance with an embodiment of the present disclosure;

FIG. 16 is a schematic view of a mapping technique in accordance with the present disclosure;

FIGS. 17-25 are perspective views of various TMS coils in accordance with the present disclosure; and

FIG. 26 is a plan view and FIG. 27 a cross-sectional view of optical imaging devices in accordance with the present disclosure.

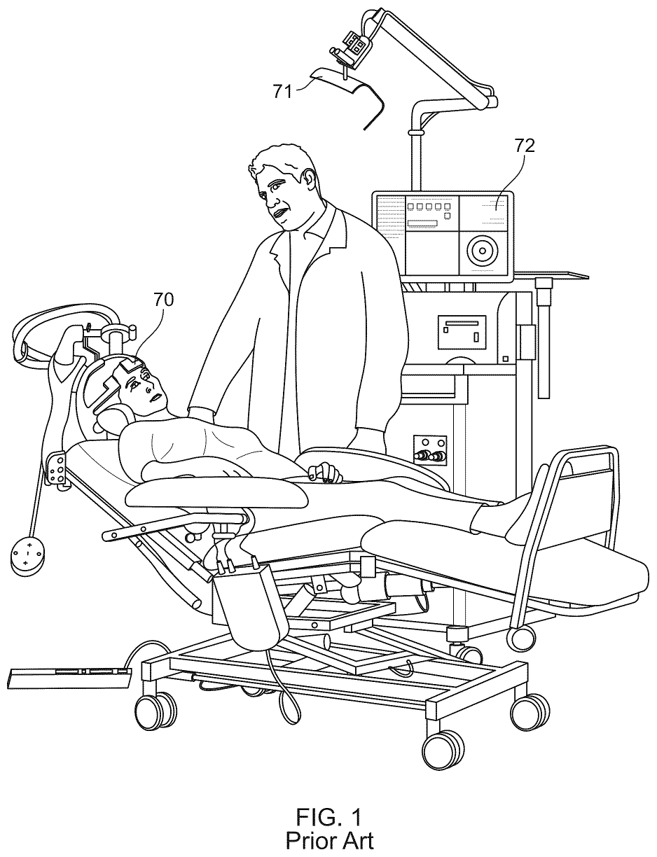

### DETAILED DESCRIPTION OF THE DISCLOSURE

As used herein the term transcranial magnetic stimulation (TMS) coil or coils shall mean the magnetic induction coils per se and their housing.

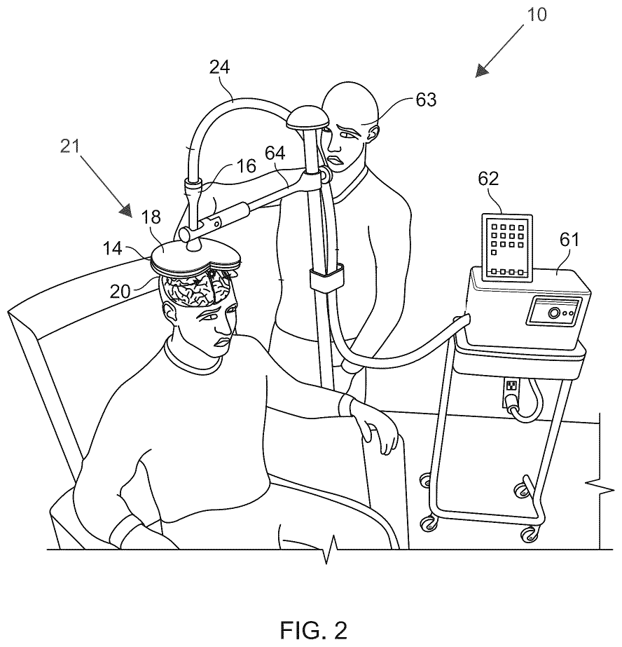

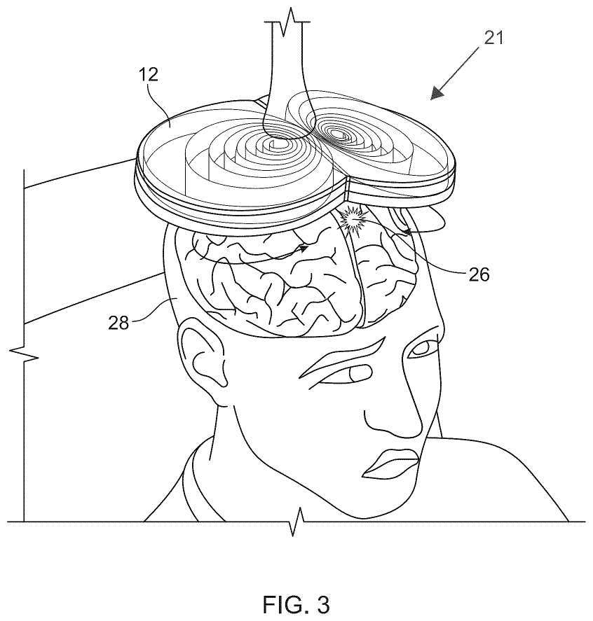

Referring to FIGS. 2-5, a neuronavigated transcranial magnetic stimulation system 10 in one embodiment includes one or more TMS coils, which itself consists of magnetic induction windings 12 in a housing 14 with leads in a cable 24. Alternatively, housing 14 may be connected to an inanimate support mechanism. Housing 14 includes a handle 16 sized for a human hand. Housing 14 includes a top surface 18 and a bottom surface 20, which can be arched to facilitate closer mating with the head of a patient.

The neuronavigated transcranial magnetic stimulation system 10 also includes a pulse generator 61 with an internal control unit and associated power source. The pulse generator sends electricity to windings 12 through a cable 24. The pulse generator 61 may be configured to communicate with smartphone, tablet or PC 62 having a program for the device to send parameters to the pulse generator 61 or for the device to receive data back from the pulse generator. The neuronavigated transcranial magnetic stimulation system 10 is designed to treat and/or ease certain symptoms by applying magnetic stimulation with a certain intensity and frequency through a patient's skull to a target area 26 in the brain within the patient's skull 28.

The coil 21 may be held in place by an operator 63, coil-holder 64, or both.

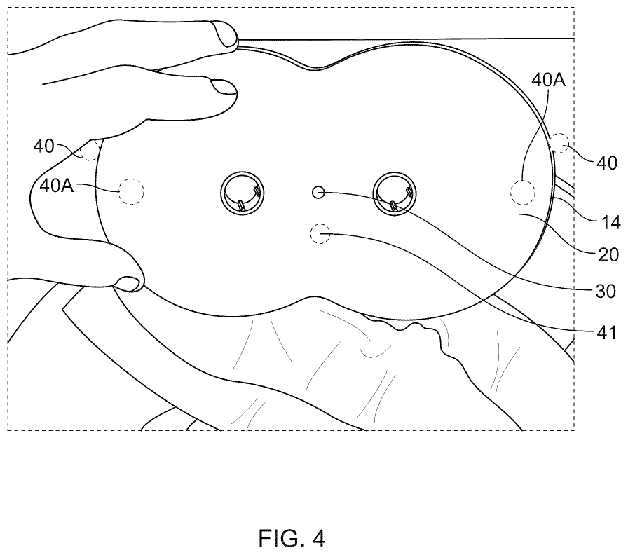

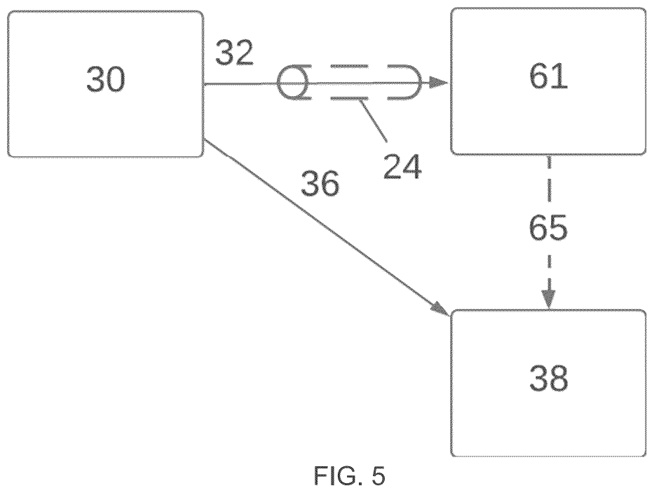

Referring in particular to FIG. 4, housing 14 includes an imaging device 30 configured to face downward from bottom surface 20, i.e., towards the head of a patient when in use, to permit direct visualization of the patient's head. In one embodiment, an imaging device 30 is located centrally relative to the magnetic induction coils. Imaging device 30 preferably comprises a camera which may be a visual light imaging camera, an ultraviolet light imaging camera or an infrared imaging camera. Referring in particular to FIG. 5, in one embodiment, imaging device 30 is connected via cable 36 to a display and memory device 38 which may be a smartphone, tablet or PC. In another embodiment, imaging device 30 is connected via a cable 32 to the pulse generator 61. Cable 32 may travel through cable 24. Optionally, pulse generator 61 may pass imaging information to device 38 which may be a smartphone, tablet or PC. This connection 65 may be wired, or wireless via Bluetooth, Wi-Fi, NFC or the like.

Alternatively, as shown in phantom at 40, the imaging devices may include spaced imaging devices located away from the center, at the sides of the magnetic induction coil's windings 12. Alternatively, two or more imaging devices shown in phantom at 40A, may be placed facing downward, away from the center of the housing, but within the housing spaced from one another at the same distance from the center of the windings 12, or attached adjacent to the edges of the windings 12.

Also, if desired, one or more contact sensors 41 configured to detect force between the coils and the patient's head may be provided, carried on the underside of housing 14. The contact sensors 41 may comprise one or more force-sensitive sensors, one or more capacitive sensors, or one or more infrared sensors.

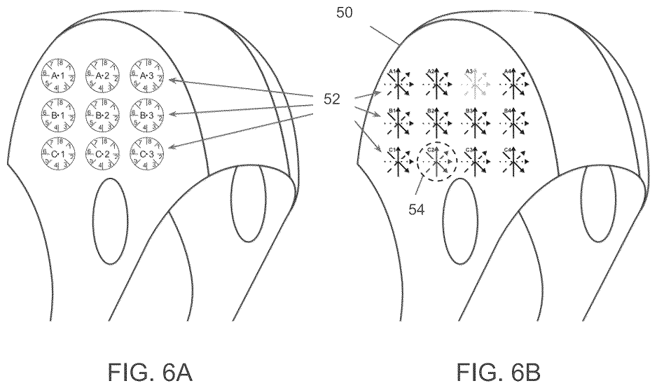

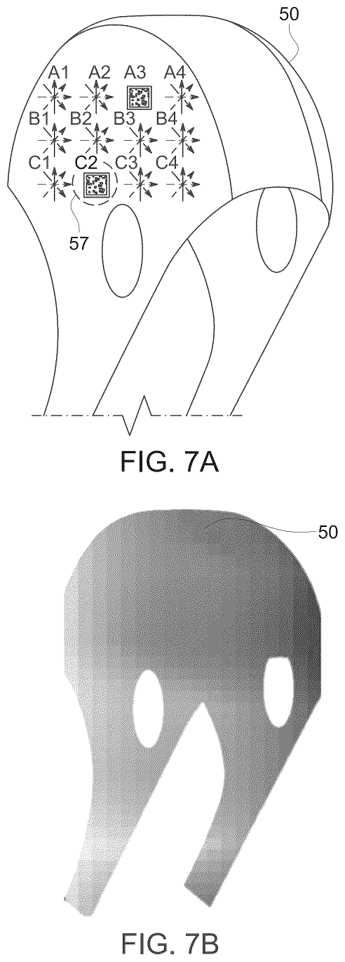

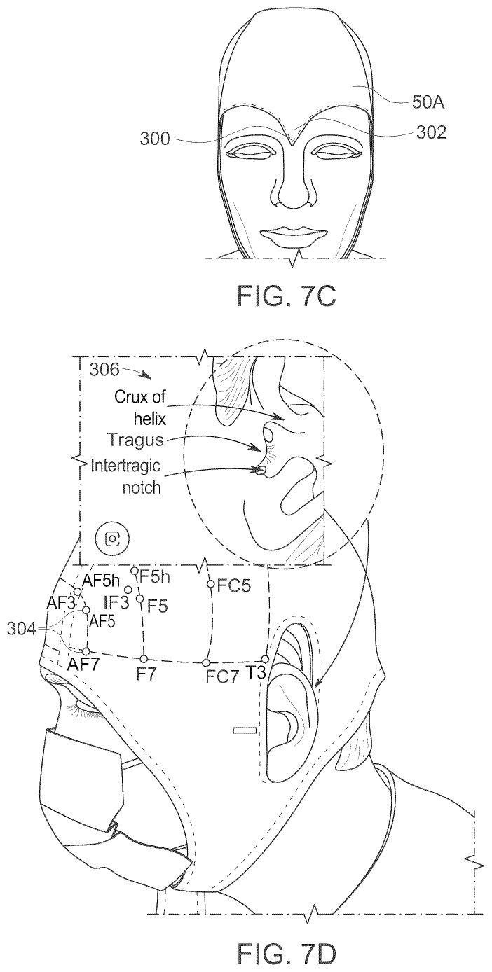

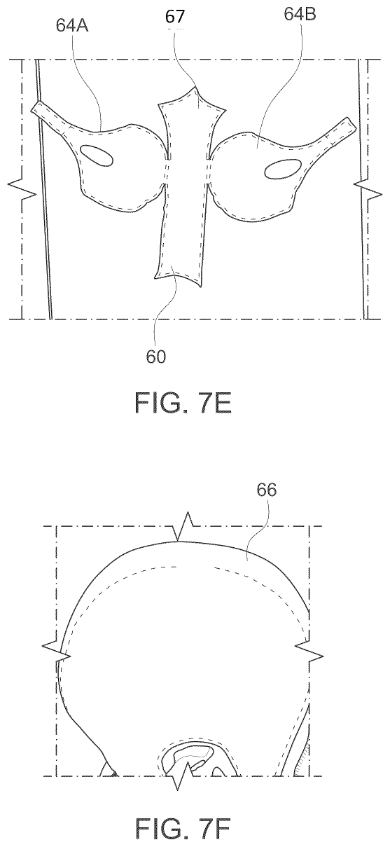

Referring also to FIGS. 6A and 6B, in a preferred embodiment of the disclosure, we provide a treatment cap 50 sized and shaped to fit snugly over a patient's head. The cap may be composed of material intentionally designed to stretch, to accommodate a defined range of head sizes slightly larger than its unstretched size. Preferably cap 50 may be provided in a kit with several different sizes to fit different size patients. Typically five sizes are sufficient to fit the majority of adult heads, a sixth size for youths, and a seventh size for small children and infants. Cap 50 includes indicia 52 in the form of a specific grid with anatomical markers printed on the cap. The indicia or markers may include text, color and/or symbols to identify specific target locations in the head of the wearer and/or shapes and patterns to indicate orientation for the TMS windings 12 to facilitate the magnetic induction in the correct location and orientation. These indicia may also be comprised of symbols, QR codes, color spectra, or any combination thereof. The indicia may be identical across numerous copies of the cap produced. Alternatively, a cap 50 may instead have unique indicia 57 in one or more locations such that the cap and any location on it can be uniquely distinguished from any other (FIG. 7A). Alternatively, the cap 50 may have printed patterns or color gradations to guide placement (FIG. 7B). The caps also may include indicia to personalize a cap to an individual patient.

Referring to FIGS. 7C and 7D, in accordance with another embodiment the present disclosure rather than use a cap with a flat brim 300, the present disclosure uses unique cap 50A geometry where the brim 300 comes to a point 302 on the midline. This point can be immediately visually confirmed to be correctly placed or not without the need for measuring tape.

As noted supra, many neurostimulation and neuroradiation techniques require precision placement of their hardware. Navigation caps are often used, with hand-drawn or pre-printed indicia on them. The hardware is then put into proper placement based on these markings either by eye or using one or more imaging devices. Unfortunately, traditional sewing techniques are highly imprecise, with seam alignment tolerances in upwards of 1 to ½ of an inch. Thus headcaps—which are universally made out of 3 or more pieces sewn together—have significant variance from cap to cap. When millimeter precision is required, this is inadequate.

In order to increase the precision in cap form, as well as the accuracy of preprinted indicia we have developed a unique system in which headcaps for neuronavigation are cut from a single 2D pattern, and then sewn together across multiple seams on both sides in order to make a 3D cap that conforms to the head. This significantly reduces alignment errors between the fabric on the sides and top of the head.

Referring to FIGS. 7E and 7F, cap 50A preferably is cut from a single piece of fabric 60 forming a center piece 67 and side pieces 64A, 64B affixed to center piece 67. The center piece 67 and side pieces 64A, 64B are then sewn together at continuous seams 66.

Additionally, we may place indicia 304 on the cap that denotes where the cap should be with respect to the tragus 306 (the point flap of skin on the ear), as an additional marker to ensure reliable fit of the cap on the head.

With both the pointed brim 300, and tragus markers 306 on both sides, these three markers may be used not only for basic visual confirmation, but an AI algorithm optionally can be implemented to ensure proper cap positioning, using a smartphone camera and slowly wave it around the patient from left to front to right side to ensure the cap is properly positioned.

Yet another feature and advantage of the instant disclosure that results from the provision of a camera central to the center of the TMS coils and a cap with indicia as above described is that we can detect coil rotation, and if desired flip or reverse the waveform polarity by changing, i.e., reversing the polarity of the power electronics to improve patient comfort.

Because the magnetic induction coil's field has a particular orientation (it is directional, not symmetric), the angle at which the magnetic induction coil is placed over a given location makes a meaningful difference in how patients experience the procedure. Specifically, even over the exact same central location, positioning the coil at different angles will activate different central and peripheral nerves. In the latter case, this may cause uncomfortable sensations at some angles, but not others. For example, at some angles, a patient's jaw may jitter during TMS, while not at others. Thus, the indicia's shape and pattern uniquely identifies each angle at which the magnetic induction coil may be placed so that, in conjunction with the camera, a viewer can see if they are properly and consistently aligned. Notably, the indicia are neither radially, nor bilaterally symmetric, and thus a rotation of 180 degrees of the magnetic induction coil will result in a different perspective on any given marker so that it is again, uniquely identified. Similarly, the text and color combination of each anatomical marking uniquely identifies the location. Locations commonly used as stimulation targets or reference locations in the therapeutic TMS community are further differentiated using color, to allow for quick and robust setup. This permits the healthcare provider to ensure that the magnetic induction windings 12 are properly positioned on the head of the wearer, and not skewed or tilted. We also can infer the coil's distance from the head of the wearer due to image size, to ensure that the coil is in full contact when seen by the imaging device.

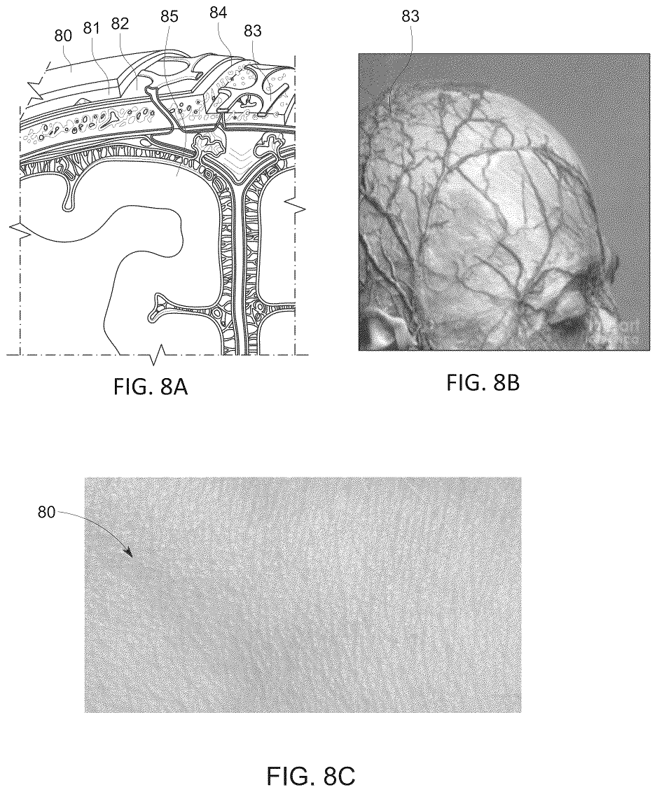

In another embodiment no specialized treatment cap is employed. Instead, patient-specific anatomical features are used to locate and maintain the coil in position. Referring to FIGS. 8A-8C, these features may comprise the epidermis 80, dermis 81 and hypodermis 82 patterns, scalp vascularization 83, bone 84 density and neural tissue configuration 85 obtained by an optical or infrared camera and/or through functional near infrared spectroscopy.

This makes it possible to perform a multi-modal “finger-print” of the precise location of the stimulation target and determine the location of the magnetic induction coil accordingly based on these individually unique anatomical features of each patient's scalp itself rather than the premarked cap. The image patterns may be recorded and saved for future treatments.

A feature and advantage of the present disclosure derives from use of one or more imaging devices internal or external to the brain energy delivery and/or detection instrument which not only ensures proper placement of the transcranial magnetic stimulation system, but also permits continuous monitoring of placement and also includes an ability to record and/or transmit placement data in real time during the entire procedure. Also, by providing target indicia 54 (FIG. 6B) on the cap, the healthcare provider can accurately locate the instrument over a target area of the brain. Alignment can be prompted via visual, auditory and/or haptic feedback.

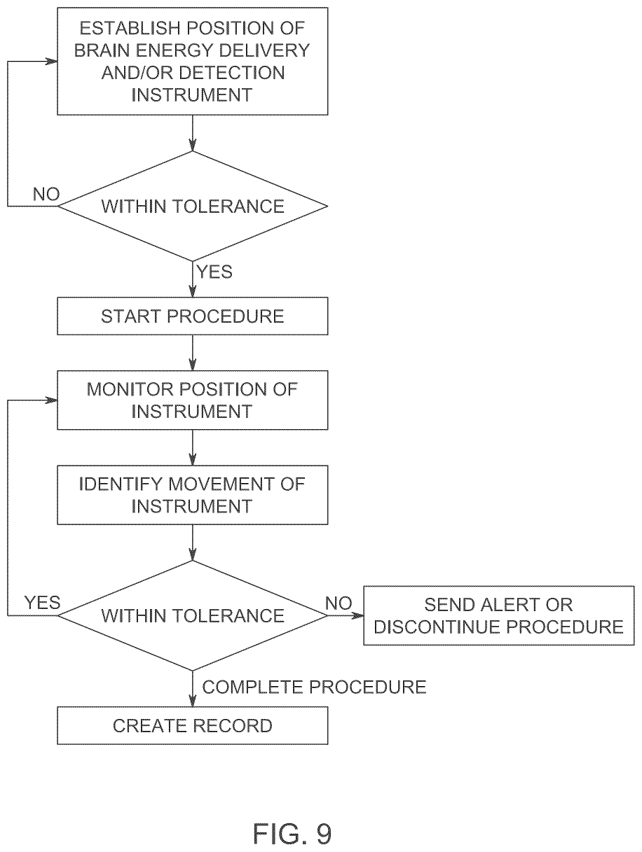

Referring to FIG. 9, another feature and advantage of the present disclosure that results from the use of built-in imaging devices is that, should the brain energy delivery and/or detection instrument be dislodged or moved (due to movement by the patient for example), an alert signal can be generated for the health care provider. Moreover, to protect the patient from possible harm, the brain energy delivery and/or detection system can be programmed to not start until it is properly positioned, and to turn off or pause delivery of stimulation pulses when misalignment of the instrument exceeds a certain tolerance, prompting correction of the position by the operator before proceeding.

A feature and advantage of the transcranial magnetic stimulation system of our disclosure is the provision of an imaging device or camera central to a center of the TMS coil head configuration, which permits direct visualization of the TMS coil(s) on the patient's head.

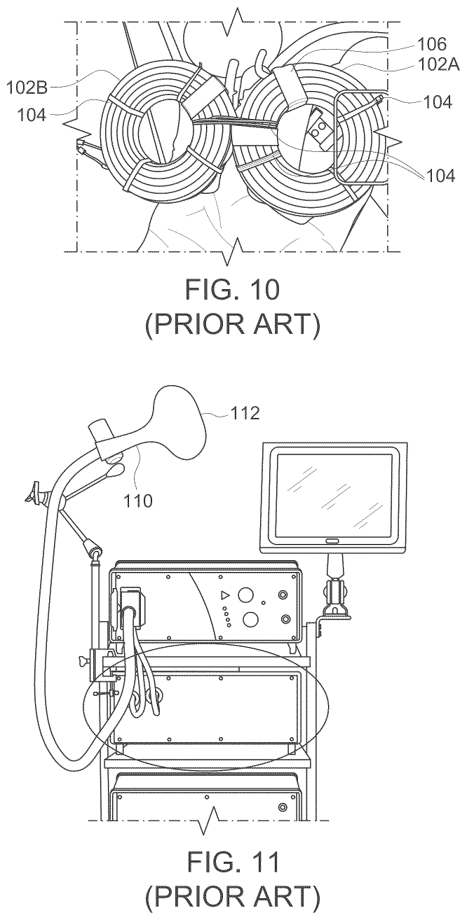

Conventional or so-called “donut” coils TMS coil head 102A, 102B employed in prior art TMS coil heads as illustrated in FIGS. 10 and 11 are geometrically unsuited for incorporation of an imaging device or camera centrally located between the coils.

In accordance with the present disclosure, we provide TMS coil configuration that permits the positioning of one or more imaging devices, i.e., one or more cameras including a single camera central to the center of the TMS coil head. However, in order to facilitate positioning a camera central to the center of the TMS coil head, we cannot simply wrap or tie our coils together as in the case of prior art traditional “donut” coils 102A, 102B using string 104 or tape 106 to bind the wires together (FIG. 10). Rather, in accordance with a preferred embodiment of the present disclosure we provide the inside of the bottom and/or top of the coil head holder with a plurality of spacers or pegs for holding the wires in place.

Conventional prior art transcranial magnetic stimulation systems also rely on circulating a coolant through the TMS coil head to dissipate excess heat and keep the TMS coil head at a proper and comfortable temperature for the patient. However, providing our novel wire coil geometry that permits us to locate an imaging device including a camera central to the center of the TMS coil head prevents cooling the TMS coil head by continuously circulating a cooling fluid through the coils. Accordingly, in accordance with the present disclosure we employ a PCM permanently packed around the wires in our TMS coil head. The PCM has sufficient thermal energy absorption capacity to cool the coils for the duration of a treatment. Accordingly, in accordance with further aspect of the disclosure, the TMS coil head is configured to be readily swapped out between treatment sessions. To facilitate this, in accordance with the present disclosure, we provide a detachable cable configured to be detachable fixed to the TMS coil head or to the TMS pulse generator. Traditional TMS coils circulate cooling fluid through a cooler so that they can be used non-stop. In accordance with the present disclosure, each TMS system is provided with multiple TMS coils so that the health care provider readily can swap out coil heads between patients. Thus, as distinguished with conventional prior art TMS coil heads that are configured to circulate coolant to the TMS coil heads and also energize the coil(s), our TMS coil head is far simpler in construction and does not need to be configured to circulate a coolant. Also, with our PCM cooled coil heads, the cable needs only a single junction, i.e., to plug into a TMS pulse generator.

In another aspect, with our PCM cooled coil heads, we provide a detachable power cable that can also detach from the TMS coil head, configured to deliver electrical power from a pulse generator to the TMS coil head. This is another advantage over conventional TMS coil heads, which require fixed and expensive cabling to both circulate coolant and deliver electrical pulses. Employing a PCM as a coolant sealed in the TMS coil head provides additional advantages. For one, the density of PCM is much lighter than traditional liquid coolants such as Galden® HT 135. As a result, our TMS coil head is lighter in weight which means we do not require TMS coil holder arms which are heavy, bulky and difficult to use (see FIG. 11).

Additionally, because our TMS coil heads are lighter in weight, we can use light weight compact ergonomically shaped handles on the TMS coil heads on the top of the coil head, as opposed to traditional TMS coil heads as illustrated in FIG. 11 that have a “paddle” handle 110 extending out from a side of the coil head 112, which forces the operator to handle the coil head like a sword which is tiring for the operator.

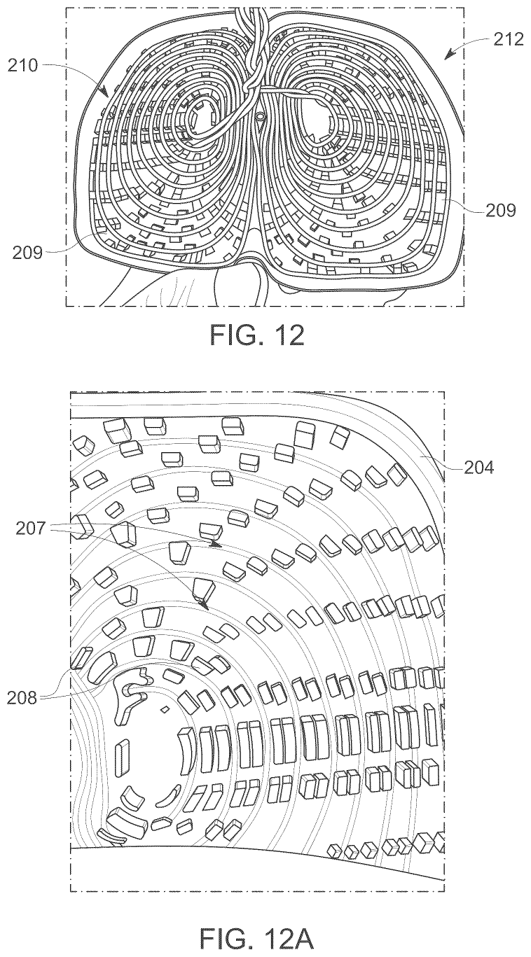

Referring to FIGS. 12, 12A, 13 and 14 a TMS coil head 200 in accordance with another embodiment of the present disclosure comprises a fluid tight housing 202 having a base 204 and a top 206. Housing 202 has a slightly concave shape for approximating the head of a patient. Base 204 includes a plurality of inlaid winding paths 207 and spacers or pegs 208 for guiding placement of the wiring within the housing. The wiring comprises the treatment coil for providing TMS magnetic stimulation to the patient's brain. The wiring includes a wiring structure comprising a continuous wire 209 wound in continuous series of wire loops 210 and 212. Wire loops 210 and 212 are essentially mirror images of one another. Wire 209 includes a conductive coil and a dielectric coating.

Wire loops 210, 212 typically are glued in position by an adhesive laid in paths 207 and/or between pegs 208, before the wire loops 210, 212 are placed into position in the base 204. Additional pegs located on and extending downwardly from the top 206 may be provided for pressing down on and holding the wire loops 210, 212 in position. Alternatively, the channels and pegs may be formed in/on the inside of the top 206, and the wire loops placed into position in the top 206. The pegs also ensure that the wire loops are spaced from one another to permit a phase change material to flow between the loops and contact the wires to increase thermal contact with the wires. The wire loops 210, 212 may be placed into position by hand or by robot. The free ends of the wire 209 is then threaded through a hole or fitting (not shown), in the top 206, and subsequently connected to a power cable which in turn is connected to a power generator of a neuronavigated transcranial magnetic stimulation system, e.g., as above described. However, the conventional circulating coil head cooling system may be bypassed since it is not needed as discussed below.

A PCM 276 such as PulseICE Organic A36 that is normally solid at ambient temperature, is then heated to melting, and the melted PCM is then poured into the bottom or top of the housing as the case may be to cover and encase the wire loops 210, 212. The PCM then is allowed to cool and solidify. PCMs have an advantage over static fluids in that they absorb much more thermal energy during the act of melting. By way of example Galden®HT 135 which traditionally has been used as a circulating heat transfer agent with conventional TMS coil heads absorbs 0.23 J/gK, whereas PulseICE Organic A36 PCM absorbs about 250 J/g just by melting. However, unlike static heat transfer agents such as Galden® HT 135, PCM's are poor conductors of heat over distance. Thus, PCM's only work when they are in close contact with a heat source. By laying the wire coils with spacing between the loops, and by solidifying the liquid PCM in situ in contact with the wire coils, in accordance with the present disclosure, we maximize thermal heat transfer from the wire coils to the PCM.

As noted supra, a preferred PCM material is PulseICE Organic A36. However, we also may mix other materials with the PCM to improve the PCM thermal conductivity, such as metal fines, e.g., of copper, tin or aluminum, carbon allotropes, e.g., graphite, or graphene, or a thermal paste. We also can affix solid heatsinks that are not very electrically conductive, e.g., aluminum oxide, to the wire coils.

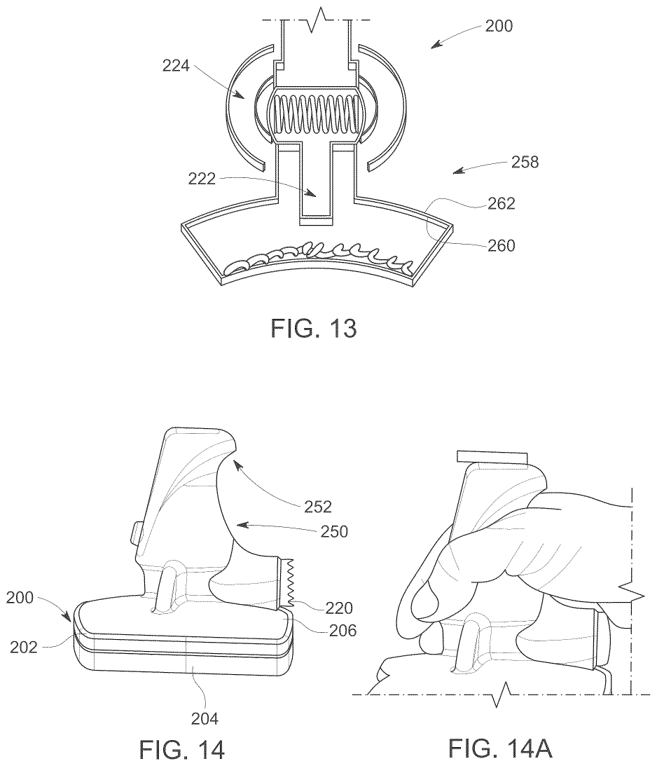

Once the PCM is solidified, the coil head is assembled, base 204 and top 206 are sealed together, a power cable 220 is attached, and the coil head 200 is ready to use. The PCM in the coil head has sufficient cooling capacity to last the length of a typical treatment, i.e., 0.5 to 10 minutes. Once treatment is complete, the coil head is allowed to cool, whereupon the PCM solidifies and is ready for reuse. Referring to FIG. 13, to speed cooling between uses, the coil head 200 may include solid heatsink elements 222 extending from the coils to the surface of the coil head 200. The coil head 200 can then be placed onto a cooling apparatus 224. In one embodiment, the cooling apparatus 224 is passive and ambient air is used to cool the heatsink elements which then transfer cooling into the PCM inside the coil head 200. In another embodiment, the cooling rate is increased by an active cooling device such as Peltier Thermocouple.

Also in order to reduce or eliminate unwanted electromagnetic interference emissions from the TMS pulse generator or the TMS coil head, rather than form the TMS pulse generator or the TMS coil head enclosure of heavy metal, we can form the TMS pulse generator or the TMS head enclosure 258 of a light weight polymeric material, and coat the inner and/or outer surfaces 260, 262 of the enclosure 258 with electrically conductive materials, such as silver, graphene, copper, carbon nanotubes and mixtures thereof.

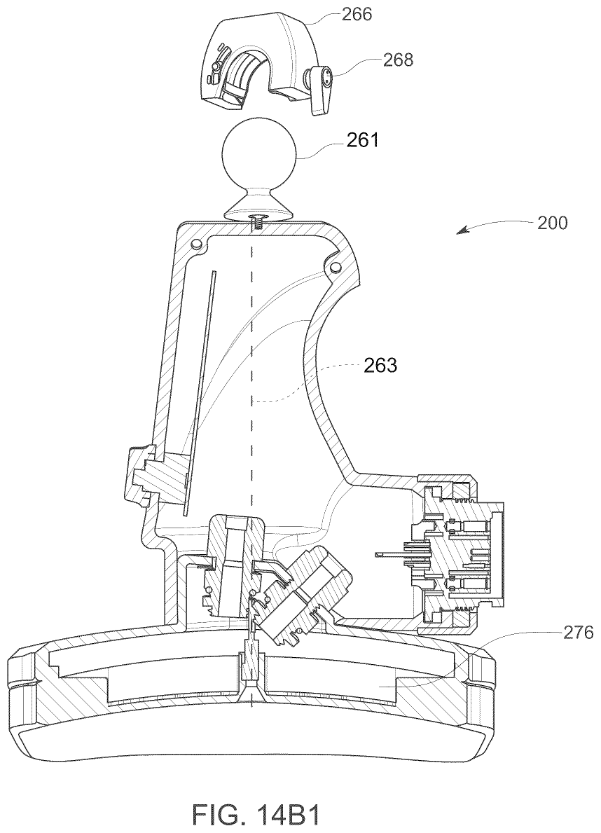

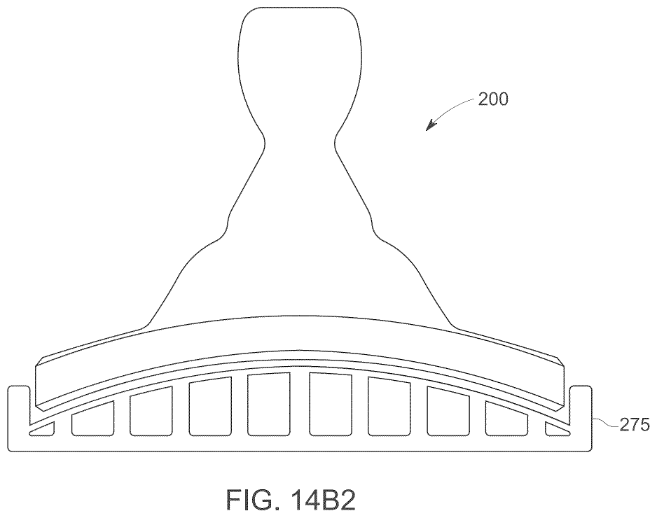

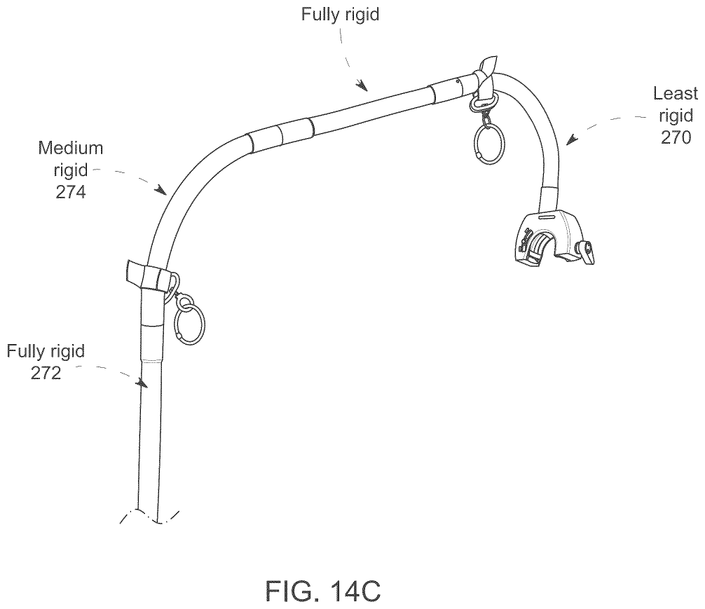

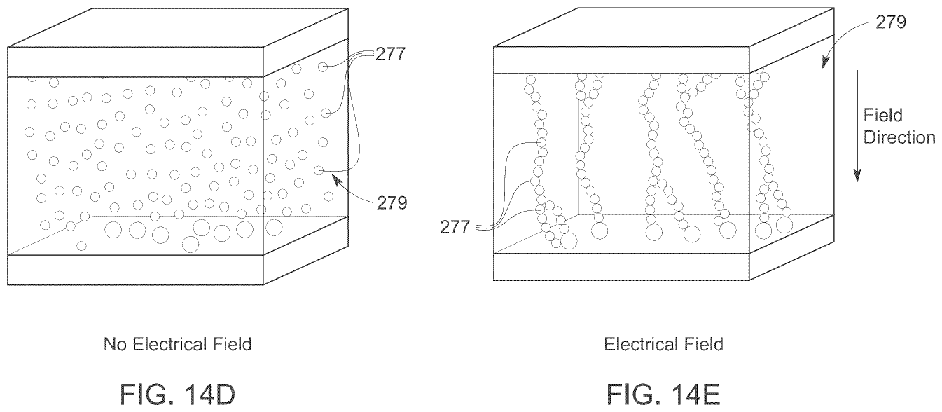

Referring also to FIGS. 14 and 14A, because the density of the PCM's we use is so much lighter than the density of traditional coolants such as Galden®HT 135, our coil head 200 is relatively light weight and easily moved by hand. To facilitate holding and moving our coil head 200, we provide our coil head 200 with a handle 250 having an overhang 252 that is ergonomically sized and shaped for the human hand. The handle also permits the operator to “free hand” the coil head 200, facilitating movement of the coil head 200 over the patient's head. The handle is both easy on the hand of the operator, but also sufficiently spaced from the wires within the coil head 200 so that the TMS pulses don't shock the operator's hand. Various changes may be made in the foregoing disclosure.

Many neurostimulation, neuroimaging, and neuroradiation techniques require precision placement of the treatment instrument. Some systems contain large apparatuses not dissimilar from small cranes to precisely hold such devices on a subject's head. These apparatuses are expensive and bulky. Other systems instead use an articulating arm, composed of 2 or more rigid pieces. These apparatuses can hold the hardware in place with a smaller footprint, but unfortunately it takes weeks to train operators to use them because it often requires manipulating and locking multiple joints at the same time, usually above a patient's head, which carry a risk of dropping the hardware on the patient's head. And still others have tried to suspend such hardware using flexible or bendable arms. However flexible or bendable arms that are made strong enough to hold the weight of the treatment instrument at distance, are undesirable as they are now so rigid as to make it very difficult to make millimeter adjustments to the position of the treatment instrument it suspends because the force required to flex or bend the arm causes overshoot of intended target, or once put over the intended target, the quite-rigid arm recoils back and causes undershoot.

The present disclosure provides a treatment instrument support system by providing a support arm that contains sections of varying selective rigidity, and in which the distal section of the support arm includes a section of least rigidity.

Referring also to FIG. 14B1, coil head 200 also may be connected to a support arm 270 (see FIG. 14C) via a ball 261 and swivel connector 266. Connector 266 preferably includes a tightening and release lever 268 configured to permit rapid tool-free swap-out and adjusting of coil heads 200. Ball 261 preferably is on a vertical axis 263 of the coil head 200.

Referring to FIG. 14B2, in yet another embodiment, coil head 200 may be cooled before or between uses, by placement in a cooling unit or cooling tray 275. Cooling tray 275 may include heatsinks to passively cool the coil head 200, or may itself be actively cooled by circulation of air and/or fluid through the cooling tray 275.

Referring to FIGS. 14C-E in accordance with another embodiment, we provide a support arm 270 comprises an elongated member having one or a plurality of sections of varying rigidity. This is accomplished by forming support arm 270 with one or more hollow flexible sections 272, 274 which are at least in part filled with an electrorheological fluid configured to reversibly change viscosity in response to an applied electromagnetic field. Electrorheological fluid comprises a suspension of fine non-conducting but electrically active particles 277 (typically up to about 50 micrometers diameter), in an electrically insulating fluid 279 (see FIGS. 14D and 14E). The apparent viscosity of these fluids can be changed reversible in response to an electric field. Changing the viscosity of the fluids in turn will change the effective rigidity of the flexible sections 272, 274 of the support arm 270, i.e., the sections containing the electrorheological fluid. As a result, we can provide a support arm 270 of varying rigidity by selectively directing an electric current across sections of the support arm 270. This permits a technician or health care provider to readily position the coil head 200 in place, and then essentially lock the coil head in position by simply activating an electrical switch.

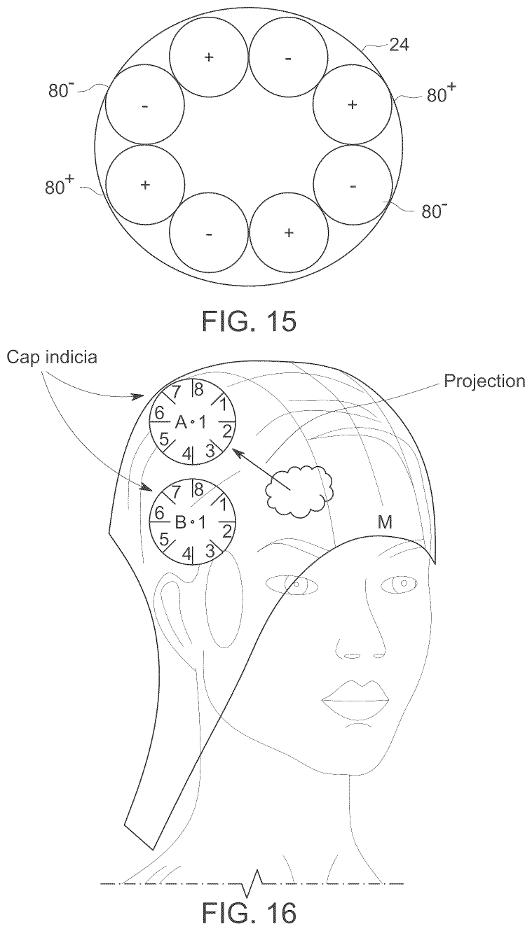

Referring to FIG. 15, the power cable 24, which may be detachable from the TMS head and/or the pulse generation, may comprise a plurality of conductors 80⁺, 80⁻, wherein half the conductors, i.e., the conductors marked with a plus “+” sign represent flowing into the TMS coil, while the conductors marked “−” represent current flowing out of the TMS coil head.

Also, in yet another aspect of the disclosure illustrated in FIG. 16, we choose what indicia on our head cap is the best location to treat a patient as follows: we give patients an MRI or fMRI in order to determine anatomically or functionally, what are the exact coordinates of the brain region to treat. However, TMS is non-invasive so we can't actually make target location within a patient's head. Accordingly, we take those coordinates (either in the head in 3d, or on the scalp in 2d) and project or map those location to indicia on our cap, and optionally with a particular rotation specified. For example, the treatment cap also may be advantageously used for directing radiation for radiation oncology treatment of the brain.

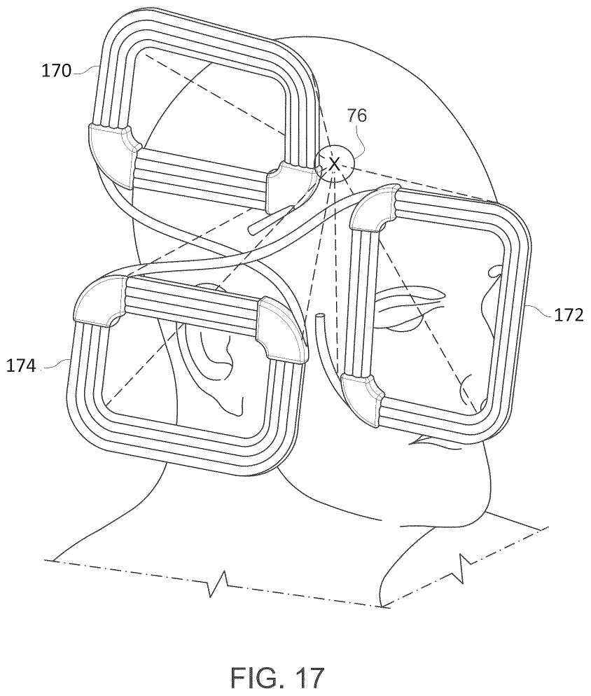

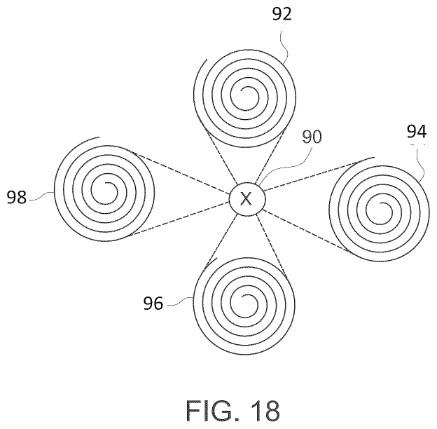

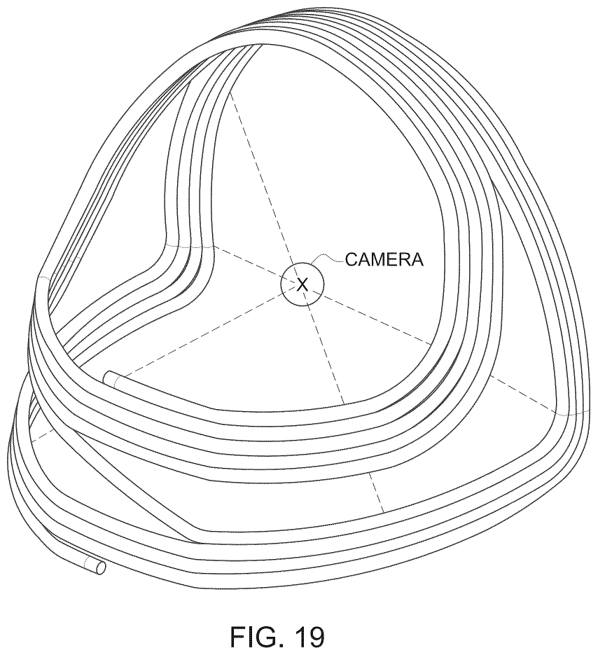

While the foregoing disclosure illustrates the advantage of employing a single imaging device between two similar coils in a two coil TMS head system, by the same token, providing an imaging device, i.e., a camera directly over the focal point of TMS coils head as above described advantageously may be employed with other coil head designs. For example, as shown in FIG. 17, a TMS coil head in accordance with the teaching of US 2016/0206896 A1 may comprise three coils 170, 172, 174 having an imaging device camera 76 located directly over the focal point of the three coils.

Referring to FIG. 18, in another embodiment, a TMS coil head may comprise four coils 92, 94, 96, 98 having a camera 90 directly over the focal point of the four coils.

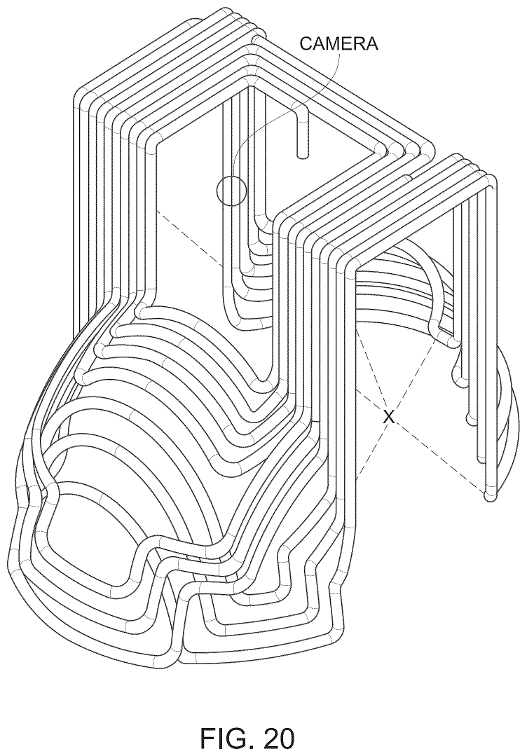

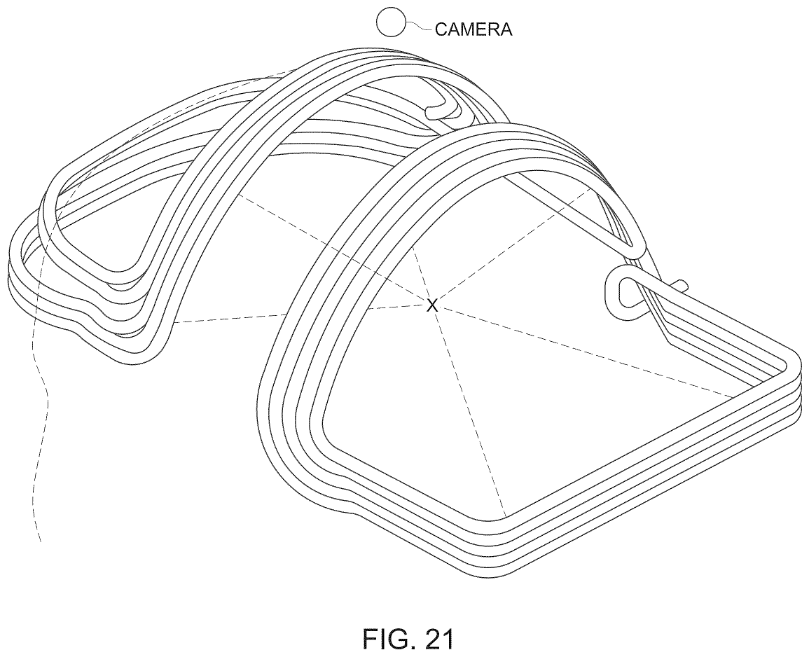

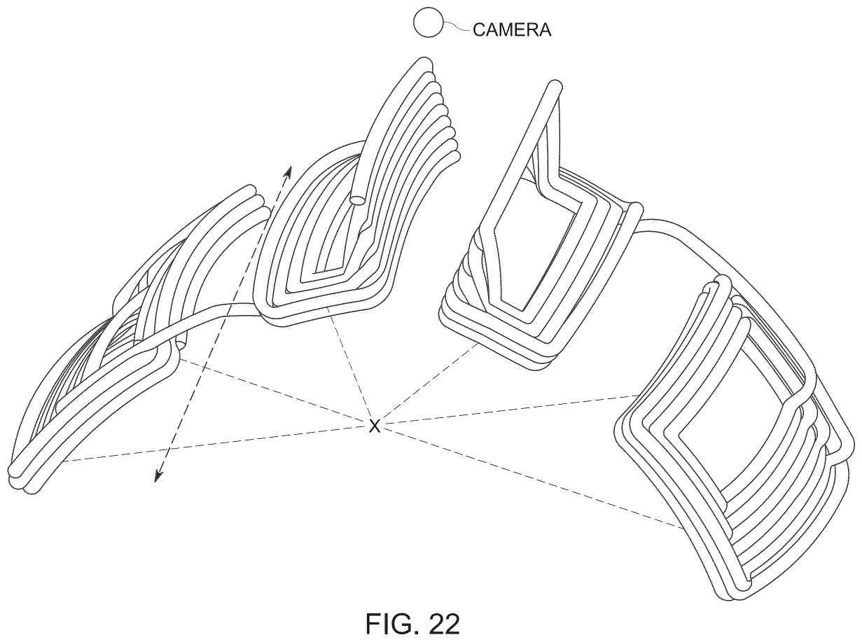

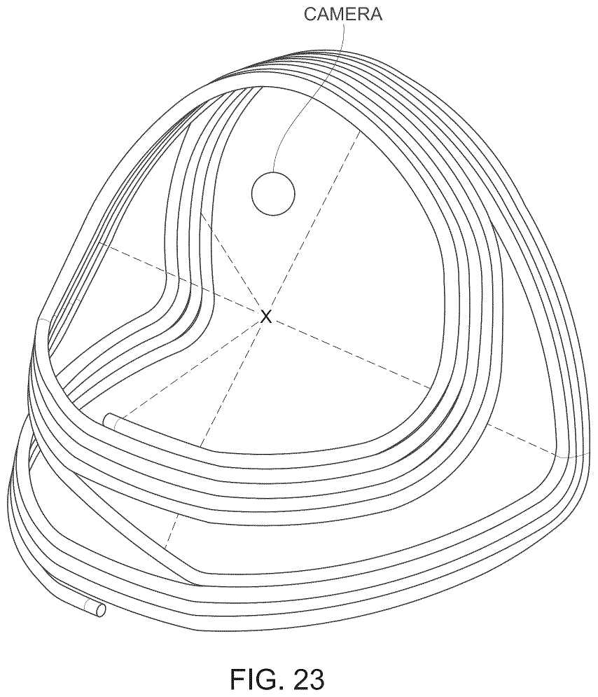

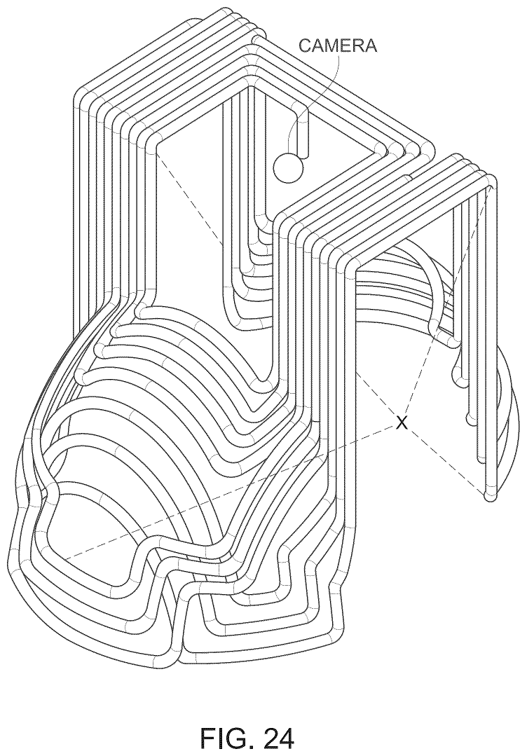

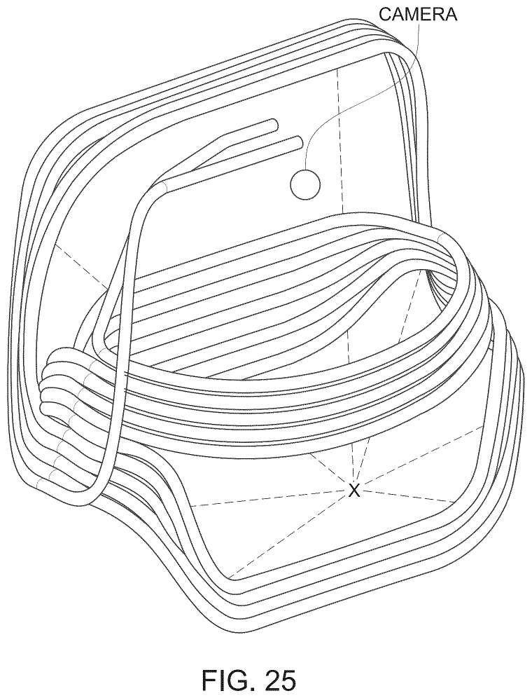

FIGS. 19-25 illustrate still other more complicated coil head geometrics taken from US Published Application Nos. US 2016/0206895 A1 (FIGS. 7 and 8), US 2016/0206896 A1 (FIGS. 11 and 13B), US 2014/0235926 A1 (FIGS. 7 and 8) and US 2014/0235927 A1 (FIG. 8), respectively, the contents of which are incorporated herein by reference, which may benefit by having a single camera directly over the focal point of the coil(s) in accordance with the subject disclosure.

While the foregoing disclosure has been directed primarily to the use of a camera positioned directly above the focal point for providing transcranial magnetic stimulation (TMS) to a patient, the disclosure advantageously may be employed with any device configured to neuronavigated devices configured to provide neurostimulation, neuroimaging, or neuroradiation whether it's pointed at indicia on a cap, or there's no cap and it's determining its position via skin texture, a marking on the head, or using IR sensing to see the vasculature, skull heterogeneities, or brain anatomy including but not limited to:
(1) Neurostimulation: A neuronavigated neurostimulation system comprising one or more imaging devices incorporated directly above a focus of stimulation and configured to permit direct visualization of one or more locations on the patient's head, a worn head cap (with or without pre-printed indicia), or the tissue below the skin (consisting of vasculature, skull, or brain tissue), where the neurostimulation technology is one or more of the following:
   i. transcranial photo biomodulation using infrared or coherent light;
   ii. transcranial focused ultrasound;
   iii. transcranial magnetic stimulation; and
   iv. transcranial electrical stimulation, and configured to cause changes to the firing patterns of neurons.
(2) Neuroimaging: A neuronavigated neuroimaging system configured to provide one or more of the following:
   a. Optical imaging, including but not limited to:
      i. Near infrared spectroscopy (NIRS), diffuse optical tomography (DOT), or optical coherence tomography (OCT), Laser speckle imaging (LSI), Photoacoustic imaging (PAI), and Laser Doppler imaging (LDI);
   b. transcranial focused ultrasound (FUS); and
   c. magnetoencephalography (MEG).
(3) Neuroradiation: A neuronavigated neuroradiation system configured to provide one or more of the following:
   i. Particle-beam (including gamma particles and electrons);
   ii. Photons from the electromagnetic spectrum including radio waves, x-rays, ultraviolet & gamma rays.
b. and configured to cause biochemical effects which include but are not limited to chemical reactions, alterations of cellular processes, alterations of tissue or cell membrane integrity, alterations in electrical or chemical communication between cells, including neurons or glia or blood vessels or abnormal tissue (e.g., tumors).

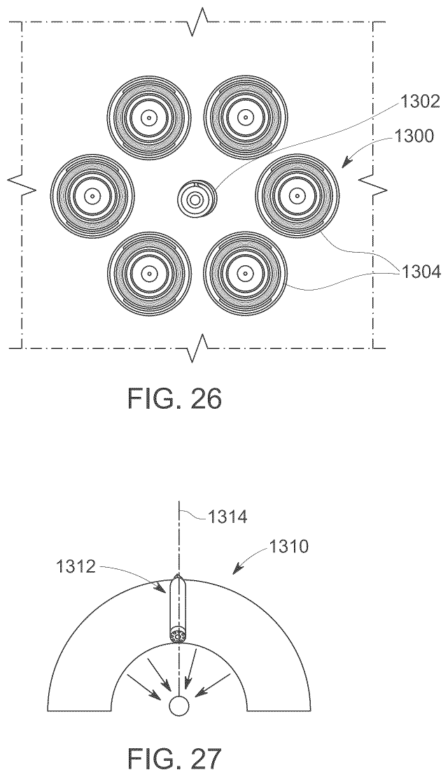

See for example, FIG. 26 in which there is illustrated a near-infrared spectroscopy device 1300 having an optical imaging device 1302 surrounded by six equispaced near-infrared emitters or detectors 1304. See also FIG. 27 in which there is illustrated an ultrasound device 1310 having an optical imaging device 1312 on the focal axis 1314 of the ultrasound device.

Various changes may be made in the foregoing disclosure. Still other changes may be made without departing from the spirit and scope thereof.

What is claimed:

1. A neuronavigated transcranial brain energy delivery and/or detection system comprising:
i) a brain energy delivery device including an energy delivery instrument configured to be placed over a target brain region of an individual's head for energy delivery; and
ii) a power generation unit for energizing the energy delivery instrument, if the brain energy delivery device requires power; and/or iii) a brain energy detection device including an energy detection instrument configured to be placed over a target brain region of an individual's head for energy detection; and
iv) a receiver unit for receiving the signal from the energy detection instrument, if the brain energy detection device requires a receiver; and
v) one or more imaging devices incorporated with the energy delivery and/or detection instrument and configured to permit direct visualization of the position of the energy delivery and/or detection instrument on an individual's head, over the target brain region.

2. The neuronavigated transcranial brain energy delivery and/or detection system of claim 1, wherein the imaging device comprises one or more cameras, selected from the group consisting of one or more visible light imaging cameras, one or more ultraviolet light imaging cameras or one or more infrared imaging cameras.

3. The neuronavigated transcranial brain energy and/or detection system of claim 1, wherein the one or more imaging devices comprise a single camera located centrally relative to the energy delivery and/or detection instrument, two or more cameras located to sides of the energy delivery and/or detection instrument, or two or more cameras located away from the center but within a housing of the energy delivery and/or detection instrument.

4. The neuronavigated transcranial brain energy delivery and/or detection system of claim 1, further comprising one or more accelerometers configured to sense orientation placement and/or changes in orientation of the energy delivery and/or detection instrument, and/or one or more contact sensors configured to detect contact and force between the energy delivery and/or detection instrument and the individual's head.

5. The neuronavigated transcranial brain energy delivery and/or detection system of claim 4, wherein the contact sensor comprises one or more force-sensitive resistors, one or more capacitive touch sensors, or one or more ultrasonic position/touch sensors.

6. The neuronavigated transcranial brain energy delivery and/or detection systems of claim 1, wherein the neuronavigated brain energy delivery and detection system is configured to regulate energy delivery and/or detection based on the instrument's position, velocity, or acceleration in any of the three translational or rotational dimensions, as inferred from measurements from embedded imaging devices or sensors, and relative to the target brain region.

7. The neuronavigated transcranial brain energy delivery and/or detection system of claim 1, further comprising one or more imaging devices external to the energy delivery and/or detection instrument, and configured to permit simultaneous visualization of the individual's head as well as the energy delivery and/or detection instrument.

8. The neuronavigated transcranial brain energy delivery and/or detection system of claim 7, wherein the imaging device comprises one or more cameras, one or more LIDAR detectors, or one or more ultrasonic detectors.

9. The neuronavigated transcranial brain energy delivery and/or detection system of claim 7, further comprising a memory device configured to create a record of the energy delivery and/or detection instrument's imaging devices, contact sensors, accelerometers, or other sensors before, during, and/or after use, which may be used to infer the instrument's position relative to the individual's head.

10. The neuronavigated transcranial brain energy delivery and/or detection system of claim 1, wherein the imaging device is configured to transmit an image of the individual's scalp vasculature, the individual's skin patterns, the individual's skull bone structure, or the individual's brain tissue.

11. The neuronavigated transcranial brain energy delivery and/or detection system of claim 1, wherein the brain energy delivery and/or detection system is selected from the group consisting of a brain stimulation system comprising:
i. transcranial photo biomodulation using infrared or coherent light;
ii. transcranial focused ultrasound;
iii. transcranial magnetic stimulation;
iv. transcranial electrical stimulation; or a brain irradiation system comprising:
V. particle-beam, including gamma particles and electrons; or
vi. photons from the electromagnetic spectrum including radio waves, x-rays, ultraviolet and gamma rays or
a brain activity sensing system comprising:
vii. an optical imaging system selected from near infrared spectroscopy (NIRS), diffuse optical tomography (DOT), or optical coherence tomography (OCT), Laser speckle imaging (LSI), Photoacoustic imaging (PAI), and Laser Doppler imaging (LDI);
viii. transcranial focused ultrasound (FUS); or
ix. magnetoencephalography (MEG); or
a brain radiation detection system comprising:
x. particle-detector, including gamma particles and electrons; or
xi. photons from the electromagnetic spectrum including radio waves, x-rays, ultraviolet and gamma rays.

12. The neuronavigated transcranial brain energy delivery and/or detection system of claim 1, further comprising a support arm configured for supporting the energy delivery and/or detection instrument, wherein the support arm comprises one or more elongated rods having one or a plurality of sections of varied rigidity, optionally containing-at least in part-an electrorheological, magnetorheological, pneumatic, or hydraulic substance configured to reversibly change rigidity in response to an electromagnetic field or by modulating the substance's quantity within the rod.

13. A neuronavigated transcranial kit comprising the neuronavigated transcranial brain energy delivery and/or detection system of claim 1, and a head cap having indicia-including target markings, text, visual texture, images, tessellations, grids, symbols, visual codes, and/or color markings-configured to overlie anatomical locations or target areas on the head of the individual, and optionally wherein the cap's geometrical features and/or specific indicia are configured to align with or emphasize distinct anatomical features of the head of the individual, allowing for continuous measurement of the cap's relative position and orientation versus the individual's head before, during, and after use, and/or wherein the markings are configured to permit continuous measurement of a relative position and orientation of the cap versus the patient's head before, during and/or after treatment, and/or further comprising a power/receiver cable, detachable from the energy delivery and/or detection instruments and configured to run between the power generator or signal receiver, and the brain energy delivery and/or brain energy detection device.

14. A method for delivering and/or detecting energy from a target brain region of an individual by directing energy onto or receiving energy from the target brain region, which method comprises:
i) providing a neuronavigated transcranial brain energy delivery and/or detection system including an energy delivery and/or detection instrument and one or more imaging devices as claimed in claim 1;
ii) positioning the energy delivery and/or detection instrument over the target region using the imaging devices to visualize the placement of the energy delivery and/or detection instrument in terms of the three translational or rotational dimensions with respect to the target region, wherein correct positioning of the energy delivery and/or detection instrument is optionally prompted by visual, auditory and/or haptic feedback; and
iii) activating and deactivating the one or more energy delivery and/or detection instruments according to a usage protocol.

15. The method of claim 14, including the step of providing an individual with a head cap having indicia—including target markings, text, visual texture, images, tessellations, grids, symbols, visual codes, and/or color markings—configured to overlie anatomical locations on the head of the individual; and
positioning the energy delivery and/or detection instrument over the target region using the imaging devices to visualize placement of the energy delivery and/or detection instrument relative to the indicia.

16. The method of claim 14, wherein the treatment comprises a treatment selected from the group consisting of a brain stimulation system comprising:
i. transcranial photo biomodulation using infrared or coherent light;
ii. transcranial focused ultrasound;
iii. transcranial magnetic stimulation;
iv. transcranial electrical stimulation; or
a brain irradiation system comprising:
V. particle-beam, including gamma particles and electrons; or
vi. photons from the electromagnetic spectrum including radio waves, x-rays, ultraviolet and gamma rays or
a brain activity sensing system comprising:
vii. an optical imaging system selected from near infrared spectroscopy (NIRS), diffuse optical tomography (DOT), or optical coherence tomography (OCT), Laser speckle imaging (LSI), Photoacoustic imaging (PAI), and Laser Doppler imaging (LDI);
viii. transcranial focused ultrasound (FUS); or
ix. magnetoencephalography (MEG); or
a brain radiation detection system comprising:
x. particle-detector, including gamma particles and electrons; or
xi. photons from the electromagnetic spectrum including radio waves, x-rays, ultraviolet and gamma rays.

17. A transcranial magnetic stimulation (TMS) system configured to be placed over a target brain region for treatment, wherein the TMS coil head comprises a housing containing one or more coil windings within the housing, and a phase change material (PCM) in contact with one or more windings within the housing, optionally with non-contiguous winding paths to maximize the PCM's contact surface area with the windings, or wherein the TMS coil head comprises a conductive cooling unit in physical contact with the TMS coil head, preferably the patient-facing surface, to dissipate heat, wherein the cooling unit functions either as a passive heatsink or actively cools using conduction, convection, or electrical cooling, preferably as a Peltier Thermocouple.

18. A method for stimulating a target brain region by transcranial magnetic stimulation, which method comprises:
i) providing a neuronavigated transcranial magnetic stimulation system including a TMS coil and one or more imaging devices as claimed in claim 1;
ii) positioning the TMS coil over the target region using the imaging devices to visualize placement of the TMS coil; and
iii) activating and/or deactivating the one or more magnetic induction coils according to a treatment protocol, and optionally including the step of providing a patient with a head cap having indicia-including target markings, text, visual texture, images, tessellations, grids, symbols, visual codes, and/or color markings-configured to overlie anatomical locations on the head of the patient; and
positioning the TMS coil over the target region using the imaging devices to visualize placement of the TMS coil relative to the indicia, wherein the correction positioning of the TMS coil optionally is prompted by visual, auditory and/or haptic feedback.

19. The method of claim 18, characterized by one or more of the following features:
(a) wherein the head cap indicia include patterns to indicate orientation of a treatment device, relative to the target area;
(b) wherein the head cap indicia are selected from the group consisting of symbols, QR codes, color spectra, text, grids, color patterns and combinations thereof;
(c) wherein the head cap includes a brim that comes to a point on a midline of the cap; and
(d) wherein the head cap is configured to denote where the cap is with respect to a tragus of the patient.
